(19) United States
(12) Patent Application Publication
(10) Pub. No.: US 2020/0181258 A1
(43) Pub. Date: Jun. 11, 2020

(54) MODIFIED ANTIBODY CONSTANT REGION

(71) Applicant: VETOQUINOL SA, Magny-Vernois (FR)

(72) Inventors: Olivier LEGER, Saint Sixt (FR); Pascale RIALLAND, Epinay Sur Orge (FR); Richard MORSE, Paris (FR)

(21) Appl. No.: 16/342,671

(22) PCT Filed: Oct. 16, 2017
(86) PCT No.: PCT/EP2017/076374

§ 371 Date: Apr. 17, 2019

(30) Foreign Application Priority Data
Oct. 17, 2016 (EP) 16194245.3

(51) Int. Cl. C07K 16/28; C12N 15/62

(52) U.S. Cl. CPC C07K 16/283; C12N 15/62; C07K 2317/20; C07K 2317/524; A61K 2039/505; C07K 2317/92; C07K 2317/94; C07K 2319/30; C07K 2317/526

(57) ABSTRACT

The present invention relates to a canine IgG Fc domain, the amino acid sequence of which comprising at least one mutation selected among:—the substitution of amino acid 15.1 of CH2 domain according to the IMGT numbering system for C-domain with tyrosine;—the substitution of amino acid 16 of CH2 domain according to the IMGT numbering system for C-domain with threonine; and—the substitution of amino acid 18 of CH2 domain according to the IMGT numbering system for C-domain with glutamic acid. The present invention also relates to an Fc-fusion protein, comprising the canine IgG Fc domain, that is genetically linked to a peptide or a protein or an engineered ligand-binding proteins or a VHH domain, and to an antibody comprising the canine IgG Fc domain.

### TECHNICAL FIELD

The present invention refers to a canine IgG Fc domain, the amino acid sequence of which comprising at least one mutation, an antibody and an Fc-fusion protein containing this canine IgG Fc domain, notably for use as a drug.

Therefore, the present invention has utility in the medical and pharmaceutical fields, especially veterinary field.

### DESCRIPTION OF RELATED ART

Monoclonal antibodies are used as therapeutics, to treat a variety of conditions including cancer, autoimmune diseases, chronic inflammatory diseases, transplant rejection, infectious diseases, and cardiovascular diseases. Currently, they are over sixty monoclonal antibodies or monoclonal antibody fragment products approved on the market, and several hundred in clinical development. Despite such acceptance and promise, there remains significant need for optimization of the structural and functional properties of antibodies.

One of the critical issues in the use of monoclonal antibodies in therapy is their persistence in the blood circulation. The rate of antibody clearance directly affects the efficacy of therapy, because it impacts the frequency and the quantity of drug administration that may cause adverse effects in the patient and also increases medical costs.

IgG is the most prevalent immunoglobulin class in humans and other mammals and is utilized in various types of immunotherapies and diagnostic procedures.

The mechanism of IgG homeostasis has been elucidated through studies related to the transfer of passive immunity from mother to foetus or neonate in rodents. Studies have found that the transport of IgG within and across polarized cells is mediated by binding of Fc region to a high-affinity Fc-receptor, named neonatal Fc receptor (FcRn).

The FcRn is a heterodimer that comprises a transmembrane α-chain with structural homology to the extracellular domains of the α-chain of major histocompatibility complex class I molecules, and a soluble light chain consisting of β₂-microglobulin (β₂-m) non convalently attached. In humans, the FcRn is expressed in placental cells, in intestinal, kidney and bronchial epithelial cells, in endothelial cells and in immune cells. FcRn binds its two major ligands, IgG and serum albumin, in a pH-dependent manner, with efficient binding at pH 6.0-6.5 and releasing at pH 7.0-7.5.

The mechanism proposed for IgG protection from catabolism is that IgGs are internalized by non-specific pinocytosis into the endosomes of the endothelial cells where the low pH promotes binding to FcRn. Bound IgG-FcRn complexes are recycled back to the cell surface and dissociate at the neutral pH of the extracellular fluid, returning to circulation in the blood. IgGs that do not bind to FcRn traffic into the lysosomes where they are degraded by proteases. According to the concentration-dependent catabolism mechanism for the survival of IgG, at low serum IgG concentrations the receptor would bind all endocytosed IgG, and efficiently return it to the circulation, yielding a long IgG half-life. Conversely, at high IgG concentrations, the receptor is saturated by IgG and a major fraction of the IgG is unbound by the receptor and traffics to be degraded, yielding a more rapid catabolism of the unbound IgG.

Various site-specific mutagenesis experiments in the Fc region of mouse IgGs have led to identification of certain critical amino acid residues involved in the interaction between IgG and FcRn. Ghetie et al. (Ghetie et al.: “Increasing the serum persistence of an IgG fragment by random mutagenesis”, Nat Biotechnol. 1997 July; 15(7):637-40 ([1])) randomly mutagenized position 252, position 254, and position 256 in a mouse IgGI Fc-hinge fragment. One mutant showed an affinity three and a half times higher for mouse FcRn at pH 6.0 and a longer serum half-life in two mouse strains, respectively, as compared to that of the wild-type Fc.

Dall'Acqua et al. (Dall'Acqua et al.: “Increasing the affinity of a human IgG1 for the neonatal Fc receptor: biological consequences”, J Immunol. 2002 Nov. 1; 169(9):5171-80 ([2])) described random mutagenesis and screening of human IgGI hinge-Fc fragment phage display libraries against mouse FcRn. They disclosed random mutagenesis of positions 251, 252, 254-256, 308, 309, 311, 312, 314, 385-387, 389, 428, 433, 434, and 436. The major improvements in IgG1-human FcRn complex stability occur in substituting residues located in a band across the Fc-FcRn interface (M252, S254, T256, H433, N434, and Y436) and to lesser extend substitutions of residues at the periphery like V308, L309, Q311, G385, Q386, P387, and N389. The variant with the highest affinity to human FcRn was obtained by combining the M252Y/S254T/T256E and H433K/N434F/Y436H mutations and exhibited an increase in affinity relative to the wild-type human IgG1.

Additionally, various publications describe methods for obtaining physiologically active molecules whose half-lives are modified either by introducing an FcRn-binding polypeptide into the molecules or by fusing the molecules with FcRn binding domains of antibodies.

Used in human medicine for thirty years, monoclonal antibodies were previously absent from treatments dedicated to animals. It is only since July 2017 that a first marketing authorization (AMM) was obtained from the European Commission for Cytopoint (lokivetmab), a caninized monoclonal antibody used in dogs to reduce symptoms caused by atopic dermatitis.

When the phylogenetic tree of the constant regions of canine, human and mouse IgG y-chains are compared, it becomes readily apparent that although there is significant sequence homology in the constant regions of subclasses within a species, there are major sequence differences between constant regions across species (Tang et al.: “Cloning and characterization of cDNAs encoding four different canine immunoglobulin gamma chains”, Vet Immunol Immunopathol. 2001 Aug. 10; 80(3-4):259-70 ([3]). This makes the identification of mutations of interest all the more arduous since it is not possible to extrapolate results obtained on one species, to another species.

In view of the constant growing of the veterinary pharmaceutical industry, and of the pharmaceutical importance of increasing the in vivo half-lives of immunoglobulins and other bioactive molecules, there is a need to develop modified IgGs and FcRn-binding fragments thereof, particularly likely to be used in veterinary pharmaceutical industry, that confer increased in vivo half-life on immunoglobulins and other bioactive molecules in an animal species.

### DESCRIPTION OF THE INVENTION

The present invention is based upon the inventors' identification of several mutations in the constant domain of a canine IgG Fc domain that increase its affinity the canine FcRn.

Accordingly, the present invention relates to variants of parent polypeptides comprising an Fc region, said variant displaying increased binding to FcRn at pH 6.0 as compared to said parent polypeptides.

Thus, a first object of the invention relates to an isolated canine IgG Fc domain, the amino acid sequence of which comprising at least one mutation selected among:
the substitution of amino acid 15.1 of CH2 domain according to the IMGT numbering system for C-domain with tyrosine,
the substitution of amino acid 16 of CH2 domain according to the IMGT numbering system for C-domain with threonine, and
the substitution of amino acid 18 of CH2 domain according to the IMGT numbering system for C-domain with glutamic acid.

Another object of the invention relates to an Fc-fusion protein comprising the canine IgG Fc domain of the invention, that is genetically linked to a moity selected among a peptide, a protein, an engineered ligand-binding proteins and a VHH domain.

Another object of the invention relates to an antibody comprising the canine IgG Fc domain of the invention.

The “IMGT numbering system” refers to the international ImMunoGeneTics database (Lefranc M et al.: “IMGT®, the international immunogenetics information system® 25 years on”. Nucleic Acids Res 2015; 43: D413-22 ([4])). It is a high quality integrated information system specializing in immunoglobulins (IG), T cell receptors (TR) and major histocompatibility complex (MHC) molecules of human and other vertebrates. The IMGT standardized description of mutations, allelic polymorphisms, 2D and 3D structure representations, is based on a unique numbering system, which can be applied to any antigen receptor, whatever the chain type or the species.

The IMGT unique numbering for all immunoglobulin and T cell receptor variable domains of all species relies on the high conservation of the structure of the variable region.

In the following description, all the amino acids are numbered according to the IMGT numbering system.

“Fc”, “Fc fragment”, “Fc region” and “Fc domain” are used herein interchangeably, includes the polypeptides comprising the constant region of an antibody excluding the first constant region immunoglobulin domain, i.e. the CH1 constant region. It therefore includes the FcRn-binding fragment. It can also be referred to as the portion of an IgG molecule that correlates to a crystallisable fragment obtained by papain digestion of an IgG molecule. Thus Fc domain comprises C.gamma.2 (CH2) and C.gamma.3 (CH3) and the hinge between C.gamma.1 (CH1) and C.gamma.2 (CH2). Although the boundaries of the Fc region may vary, the human IgG heavy chain Fc region is usually defined to comprise residues 231 to 447 or 244 to 478 according to the Eu and Kabat numbering respectively to its carboxyl-terminus. In the following, the numbering is according to the IMGT numbering system (Lefranc M-P. “Unique database numbering system for immunogenetic analysis”. Immunol Today 1997; 18:509 ([5])).

By “variant”, “mutated” or “modified” as used herein is meant a polypeptide sequence that differs from that of a parent polypeptide sequence by virtue of at least one amino acid modification.

By “parent polypeptide” as used herein is meant an unmodified polypeptide comprising or consisting of an Fc region that is subsequently modified to generate a variant. Said parent polypeptide may be a naturally occurring polypeptide, or a variant of a naturally occurring polypeptide, or an engineered version of a naturally occurring polypeptide, or a synthetic polypeptide. Parent polypeptide may refer to the polypeptide itself, or the amino acid sequence that encodes it. In the context of the present invention, the parent polypeptide comprises an Fc region selected from the group of wild-type canine Fc regions, their fragments and their mutants. Accordingly, the parent polypeptide may optionally comprise pre-existing amino acid modifications in its Fc region as compared to wild-type Fc regions.

Advantageously, the parent polypeptide may be an antibody, an immunoglobulin, an Fc fusion polypeptide, an Fc conjugate, this list not being limitative. Accordingly, by “parent immunoglobulin” as used herein is meant immunoglobulin polypeptide that is subsequently modified to generate a variant immunoglobulin, and by “parent antibody” as used herein is meant antibody that is subsequently modified to generate a variant antibody. It should be noted that “parent antibody” includes, but are not limited to, known commercial, recombinantly produced antibodies.

The modified canine IgG Fc domain of the invention may comprise one, or two, or three, or four, or five, or six, or seven mutations compared to the parent polypeptide, selected among those described above.

According to the invention, the mutation comprising the 3 substitutions, i.e. the substitution of amino acid 15.1 of CH2 domain with tyrosine, the substitution of amino acid 16 of CH2 domain with threonine, and the substitution of amino acid 18 of CH2 domain with glutamic acid, is also referred herein as “YTE” mutation.

According to the invention, the canine IgG Fc domain of the invention may have an increased binding affinity for the canine FcRn compared to the corresponding parent canine IgG Fc domain that does not comprise the amino acid residue mutation(s).

The binding affinity of the canine IgG Fc domain may be increased by at least 1.2 or at least 1.5, or at least 2, or least 3, or at least 4, or at least 5 fold, or more, compared to the binding affinity of the corresponding wild type canine IgG Fc domain, i.e. a canine IgG Fc domain that does not have the mutation(s) according to the invention.

The relative affinity of the canine IgG Fc domain for FcRn can be evaluated by well-known methods of the prior art. For example, the one skilled in the art may determine the molecular dynamics of the contact residues between the Fc and FcRn using in silico bioinformatics tools or he may determine the dissociation constant (Kd) using Surface Plasmon Resonance (SPR) experiments as illustrated in the Example 2 of the present application. If the variant has a Kd 1.2 fold lower than that of its corresponding parent then the said variant is an optimized variant according to the invention.

The term “in vivo half-life” as used herein refers to the body's cleansing to eliminate the polypeptide from the body, which can result from an increased serum half-life and/or a decreased renal clearance and/or an increased MRT (mean residence time). The in vivo half-life may be calculated by any suitable method known by the person skilled in the art, for example the enzyme linked immunosorbent assay (ELISA) procedure for measuring plasma antibody titers.

According to the invention, the binding affinity of the canine IgG Fc domain of the invention may be increased at a pH of 6 compared to the parent polypeptide.

In order to increase the retention of the Fc region in vivo, the increase in binding affinity for FcRn must occur at around pH 6, while maintaining loss of affinity at around pH 7.4. Fc regions are believed to have a longer half-life in vivo, because the binding to FcRn at pH 6.0 allows the sequestration of Fc regions into endosomes. FcRn in acidic binds to IgG internalized through pinocytosis, recycling it to the cell surface and releasing it at the basic pH of blood, thereby preventing IgG from undergoing lysosomal degradation. Therefore, amino acid modifications in the Fc region that increase FcRn binding at the lower pH while still allowing release of Fc region at higher pH, ideally increase Fc regions' half-life in vivo.

The parent polypeptide comprising an Fc region and the variant polypeptide of the invention are canine IgG Fc domains. “canine” refers herein to the Canidae family, and includes more particularly dogs, wolves, jackals, foxes, coyotes. The canine IgG Fc domain of the invention may be selected among those of dog IgG2 (i.e. having chain B), dog IgG3 (i.e. having chain C), and dog IgG4 (i.e. having chain D). Preferably, the canine IgG Fc domain of the invention is selected among dog IgG2 or dog IgG4.

The canine IgG Fc domain may be a dog (Canis lupus familiaris) IgG Fc domain.

The canine IgG Fc domain may comprise an amino acid sequence selected among SEQ ID NO. 1 and SEQ ID NO. 2, corresponding respectively to YTE mutant dog IgG2 and YTE mutant dog IgG4:

SEQ ID NO: 1:
APEMLGGPSVFIFPPKPKDTLYITREPEVTCVVVDLDPEDPEV
QISWFVDGKQMQTAKTQPREEQFNGTYRVVSVLPIGHQDWLKG
KQFTCKVNNKALPSPIERTISKARGQAHQPSVYVLPPSREELS
KNTVSLTCLIKDFFPPDIDVEWQSNGQQEPESKYRTTPPQLDE
DGSYFLYSKLSVDKSRWQRGDTFICAVMHEALHNHYTQESLSH
SPGK
SEQ ID NO: 2:
VPESLGGPSVFIFPPKPKDILYITREPEITCVVLDLGREDPEV
QISWFVDGKEVHTAKTQPREQQFNSTYRVVSVLPIEHQDWLTG
KEFKCRVNHIGLPSPIERTISKARGQAHQPSVYVLPPSPKELS
SSDTVTLTCLIKDFFPPEIDVEWQSNGQPEPESKYHTTAPQLD
EDGSYFLYSKLSVDKSRWQQGDTFTCAVMHEALQNHYTDLSLS
HSPGK

In an embodiment of the invention, the amino acid sequence of the canine IgG Fc domain of the invention may comprise:
the substitution of amino acid 15.1 of CH2 domain according to the IMGT numbering system for C-domain with tyrosine,
the substitution of amino acid 16 of CH2 domain according to the IMGT numbering system for C-domain with threonine, and
the substitution of amino acid 18 of CH2 domain according to the IMGT numbering system for C-domain with glutamic acid.

In one embodiment, the polypeptide variants of the invention may be selected from the group consisting of Fc-fusion protein variants and Fc-conjugate variants. Fc-fusion protein and Fc-conjugates consist of an Fc region linked to a partner. The Fc region can be linked to its partner with or without a spacer.

According to the present invention, an Fc fusion protein is a protein comprising a protein, a polypeptide or a small peptide linked to an Fc region. The Fc fusion protein may optionally comprise a peptide spacer. Virtually any protein or small molecule may be linked to Fc regions to generate an Fc fusion. The invention also relates to conjugated polypeptides, such as translated proteins, polypeptides and peptides that are linked to at least one agent to form a modified protein or polypeptide. According to the present invention, an Fc conjugate results from the chemical coupling of a Fc region with a conjugate partner. The coupling reaction generally uses functional groups on the Fc region and on the conjugate partner. Various linkers are known in the art to be appropriate for the synthesis of conjugate; for example, homo-or hetero-bifunctional linkers, amino acids such as selectively-cleavable linkers, synthetic linkers, or other amino acid sequences may be used to separate proteinaceous moieties.

Protein fusion partners or conjugate partners may include, but are not limited to, the variable region of any antibody, a polypeptide derived from a variable region of any antibody, a VHH domain (also named single heavy chain domain or Nanobody®), the target-binding region of a receptor, an adhesion molecule, a ligand, an enzyme, a cytokine, a chemokine, or some other protein or protein domain, for example DARPins or Ankyrin repeat proteins and Anticalin proteins. In particular the Fc-fusion protein can be an immunoadhesin i.e antibody-like protein which combines the binding domain of a heterologous “adhesion” protein (i.e receptor, ligand or enzyme) with a fragment of immunoglobulin constant domain (i.e. an Fc region). Small peptide fusion partners may include, but are not limited to, any therapeutic agent that directs the Fc fusion to a therapeutic target. Such targets may be any molecule, preferably an extracellular receptor that is implicated in disease.

Suitable conjugate partners may also include, but are not limited to, therapeutic polypeptides, labels (for example of labels, see further below), drugs, for example anti-inflammatory drugs, cytotoxic agents, cytotoxic drugs (e.g., chemotherapeutic agents and anti-tumor agents), toxins and active fragments of such toxins, therapeutic enzymes, radio-labeled nucleotides, antiviral agents, chelating agents, cytokines, growth factors, and oligo- or poly-nucleotides, and reporter molecules defined as any moiety that may be detected using an assay, such as enzymes, radiolabels, haptens, fluorescent labels, phosphorescent molecules, chemiluminescent molecules, chromophores, luminescent molecules, photoaffinity molecules, colored particles or ligands, such as biotin. Suitable toxins and their corresponding fragments include, but are not limited to, diptheria A chain, exotoxin A chain, ricin A chain, abrin A chain, curcin, crotin, phenomycin, enomycin and the like. A cytotoxic agent may be any radionuclide which can be directly conjugated to the Fc variant or sequestrated by a chelating agent which is covalently attached to the Fc variant. In additional embodiments, the conjugate partners can be selected from the group comprising corticoides calicheamicin, auristatins, geldanamycin, maytansine, and duocarmycins and analogs.

Such variants of interest may have an increased binding to FcRn at lowered pH (e.g. at about pH 6), and substantially unmodified binding at higher pH (e.g. at about pH 7.4). Advantageously, the Fc-fusion protein and Fc-conjugate variants display increased in vivo half-lives as compared to parent polypeptides.

The antibody of the invention comprises a canine IgG Fc domain as defined above.

In a preferred embodiment, the polypeptide variant of the present invention is a variant antibody of a parent antibody. The term “antibody” is used herein in the broadest sense. According to the present invention, “antibody” refers to any polypeptide which at least comprises (i) a Fc region and (ii) a binding polypeptide domain derived from a variable domain of an immunoglobulin. The said binding polypeptide domain is able to bind specifically one given target antigen or a group of target antigens. A binding polypeptide domain which derives from a variable region of an immunoglobulin comprises at least one or more CDRs. Herein, antibodies include, but are not limited to, full-length immunoglobulins, monoclonal antibodies, a VHH domain (also named single heavy chain domain or) Nanobody®, multi-specific antibodies, Fc-fusion protein comprising at least one variable region, synthetic antibodies (sometimes referred to herein as “antibody mimetics”), chimeric antibodies, caninized antibodies and fully canine antibodies. Antibodies also encompass antibody-fusion proteins, antibody conjugates and fragments of each respectively. Accordingly a variant antibody of the invention comprises, in its Fc region, at least one amino acid modification or combination of modifications above- cited that increase its binding affinity for FcRn as compared to its parent antibody. Of particular interest are antibody variants that display increased binding affinity to FcRn at lowered pH (e.g at about pH 6), and have substantially unmodified binding at higher pH (e.g. at about pH 7.4). Furthermore, of particular interest are antibody variants which have increased in vivo half-lives as compared to parent polypeptides. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Harlow et al.: “Antibodies: A Laboratory Manual”, Cold Spring HarborLaboratory Press, 2nd ed. 1988 ([6])); Hammerling, et al.: “Monoclonal Antibodies and T-Cell Hybridomas”, Elsevier, N. Y., 1981, pp. 563-681 ([7]). The term “monoclonal antibody” as used herein is not limited to antibodies produced through hybridoma technology, and refers to an antibody that may be derived from a single B cell, a single clone, including any eukaryotic, prokaryotic, or phage clone, and not the method by which it is produced.

Methods for producing and screening for specific antibodies using hybridoma technology are routine and well known in the art. In a non-limiting example, mice can be immunized with an antigen of interest or a cell expressing such an antigen.

In one embodiment, a variant antibody of the invention is selected from the group consisting of variants of parent full-length antibodies. By “full length antibody” herein is meant the structure that constitutes the natural biological form of an antibody, including variable and constant regions. The parent polypeptide of a full-length antibody variant of the present invention can be a wild-type antibody, a mutant of a wild-type antibody (e.g. comprising pre-existing modifications), an engineered version of a wild-type antibody, this list not being limitative. The structure of a full-length antibody is generally a tetramer. Each tetramer is typically composed of two identical pairs of polypeptide chains, each pair having one “light” (typically having a molecular weight of about 25 kDa) and one “heavy” chain (typically having a molecular weight of about 50-70 kDa).

Examples of full-length antibodies are immunoglobulins which encompass dog IgG2 (chain B), dog IgG3 (chain C), and dog IgG4 (chain D) classes.

In preferred embodiments, the said full-length antibody variant is selected from the group consisting of variants of IgGs.

Of particular interest are antibodies that comprise (a) a Fc variant of the inventions, and (b) one of the following binding polypeptide domains derived from a variable region of an immunoglobulin (i.e. which comprise at least one CDR): (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) isolated CDR regions, (v) F(ab′)2 fragments, a bivalent fragment comprising two linked Fab fragments (vi) single chain Fv molecules (scFv), wherein a VH domain and a VL domain are linked by a peptide linker which allows the two domains to associate to form an antigen binding site, (vii) bispecific single chain Fv and (viii) “diabodies” or “triabodies”, multivalent or multispecific fragments constructed by gene fusion, this list not being limitative.

In another embodiment, the antibody is a minibody. Minibodies are minimized antibody-like proteins comprising a scFv joined to a CH3 domain. In some cases, the scFv can be joined to a full-length Fc region, and may also include the hinge region or fragment thereof. In one embodiment, the antibodies of the invention are selected from the group of multispecific antibodies, and notably from the group of bispecific antibodies which are sometimes referred to as “diabodies”. These antibodies bind to two (or more) different antigens. Diabodies can be manufactured in a variety of ways known in the art, e.g., chemically prepared or derived from hybridomas.

In one embodiment, the said antibody variant is a fully canine antibody with at least one amino acid modification as outlined herein. “Fully canine antibody” refers to an antibody entirely comprising sequences originating from canine genes. In some cases this may be canine antibodies that have the gene sequence of an antibody derived from a canine chromosome with the modifications outlined herein.

Covalent modifications of antibodies are also included within the scope of this invention, and are generally, but not always, done post-translationally. Such modifications include, but are not limited to, glycosylates, labelling and conjugation. The term “labeling group” means any detectable label, which is a compound and/or element that can be detected due to its specific functional properties, and/or chemical characteristics, the use of which allows the antibody to which it is attached to be detected, and/or further quantified if desired. In some embodiments, the labeling group is coupled to the antibody via spacer arms of various lengths to reduce potential steric hindrance. Various methods for labeling proteins are known in the art and may be used in performing the present invention. In general, labels fall into a variety of classes, depending on the assay or on the diagnostic procedure in which they are to be detected: a) isotopic labels, which may be radioactive or heavy isotopes; b) magnetic labels (e.g., magnetic particles); c) redox active moieties; d) optical dyes; enzymatic groups (e.g. horseradish peroxidase, β-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.).

Specific labels include optical dyes, including, but not limited to, chromophores, phosphors and fluorophores, with the latter being specific in many instances. Fluorophores can be either fluorescent “small molecules” fluorescent, or fluorescent proteins. In another embodiment, the antibody variants of the present invention may be fused to or conjugated to a protein or a small molecule which are not used as a labelling group as described above. Virtually any protein or small molecule may be linked to an antibody. Protein fusion partners may include, but are not limited to, the target-binding region of a receptor, an adhesion molecule, a ligand, an enzyme, a cytokine, a chemokine, or some other protein or protein domain. Small molecules include, but are not limited to drugs, cytotoxic agents (e.g., chemotherapeutic agents), toxins or active fragments of such toxins.

The antibody and the Fc-fusion protein of the invention may have an increased in vivo half-life compared to the corresponding wild type or parent antibody or Fc-fusion protein, which can be an increase of at least 1.5, or at least 2, or least 3, or at least 4, or at least 5 fold, or more, compared to the in vivo half-life of the corresponding wild type or parent antibody or Fc fusion protein, i.e. an antibody or Fc fusion protein that does not have the mutation(s) of the canine IgG Fc domain according to the invention.

This is advantageously due to an increased binding affinity at pH 6.0 of the canine IgG Fc domain of the invention for the canine FcRn compared to parent canine IgG Fc domain.

Another object of the invention relates to a nucleic acid encoding a canine IgG Fc domain of the invention, or an antibody of the invention, or an Fc-fusion protein of the invention.

Another object of the invention relates to an expression vector having the nucleic acid of the invention.

Another object of the invention relates to a stable cell line producing a canine IgG Fc domain of the invention, or an antibody of the invention, or an Fc-fusion protein of the invention, having the expression vector of the invention.

An “isolated” nucleic acid molecule is one which is separated from other nucleic acid molecules which are present in the natural source of the nucleic acid molecule. Moreover, an isolated nucleic acid molecule, such as a cDNA molecule, can be substantially free of other cellular material, or culture medium when produced by recombinant techniques, or substantially free of chemical precursors or other chemicals when chemically synthesized. An isolated nucleic acid molecule does not include cDNA molecules within a cDNA library. In a preferred embodiment of the invention, nucleic acid molecules encoding antibodies are isolated or purified. In another preferred embodiment of the invention, nucleic acid molecules encoding fusion proteins are isolated or purified.

The stable cell line producing the canine IgG Fc domain, or the antibody, or the Fc-fusion protein of the invention, and having the expression vector as defined above, may be selected from the group consisting of SP2/0, YB2/0, IR983F, Namalwa human myeloma, PERC6, CHO-DG44, CHO-DUK-B11, CHO-K-1, CHO-Lec10, CHO-Lec1, CHO-Lec13, CHO Pro-5, CHO/DHFR-, Wil-2, Jurkat, Vero, Molt-4, COS-7, 293-HEK, BHK, K6H6, NSO, SP2/0-Ag 14 and P3X63Ag8.653.

Nucleic acid encoding the canine IgG Fc domain of the invention, the antibody of the invention, or the Fc-fusion protein of the invention can be obtained by standard molecular biology or biochemistry techniques, such as DNA chemical synthesis, PCR amplification or cDNA cloning and can be inserted into expression vectors such that the genes are operatively linked to transcriptional and translational control sequences. In this context, the term “operatively linked” is intended to mean that a gene to express is ligated into a vector such that transcriptional and translational control sequences within the vector serve their intended function of regulating the transcription and translation of the gene. The expression vector and expression control sequences are chosen to be compatible with the expression host cell used. In case of an antibody or of an Fc-fusion protein, the gene of the IgG Fc domain of the invention and the other part of the antibody or protein can be inserted into separate vector or, alternatively, both genes are inserted into the same expression vector.

The genes may be inserted into the expression vector by standard methods, such as ligation of complementary restriction sites on the gene fragment and vector. The IgG Fc domain of the invention can be used to create full-length antibody genes by inserting them into expression vectors already encoding Fab region of the desired sequence. Additionally or alternatively, the recombinant expression vector can encode a signal peptide that facilitates secretion of the canine IgG Fc domain, or the antibody, or the Fc-fusion from a host cell. The gene can be cloned into the vector such that the signal peptide is linked in frame to the amino terminus of the gene. The signal peptide can be an immunoglobulin signal peptide or a heterologous signal peptide, such as a signal peptide from a non-immunoglobulin protein.

In addition to the genes, the recombinant expression vectors of the invention may carry regulatory sequences that control the expression of the genes in a host cell. The term “regulatory sequence” is intended to include promoters, enhancers and other expression control elements, such as polyadenylation signals that control the transcription or translation of the antibody chain genes. Regulatory sequences for mammalian host cell expression include viral elements that direct high levels of protein expression in mammalian cells, such as promoters and/or enhancers derived from cytomegalovirus (CMV), Simian Virus 40 (SV40), adenovirus such as the adenovirus major late promoter (AdMLP), and polyoma. Alternatively, nonviral regulatory sequences may be used, such as the ubiquitin promoter or P-globin promoter. Still further, regulatory elements composed of sequences from different sources, such as the SRa promoter system, which contains sequences from the SV40 early promoter and the long terminal repeat of human T cell leukemia virus type 1.

In addition to the antibody chain genes and regulatory sequences, the recombinant expression vectors of the invention may carry additional sequences, such as sequences that regulate replication of the vector in host cells, such as origins of replication, and selectable marker genes for facilitating selection of host cells into which the vector has been introduced. For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neo gene (for G418 selection).

Another object of the invention relates to an in vitro process for producing a canine IgG Fc domain of the invention, or an antibody of the invention, or an Fc-fusion protein of the invention, comprising the steps of:

(A) providing a host cell with an expression vector having a nucleic acid of the invention, under conditions such that the host cell expresses said nucleic acid, and,
(B) collecting said antibody constant region or said antibody produced by the host cell.

The term “host cell” as used herein refers to the particular subject cell transfected with a nucleic acid molecule or infected with phagemid or bacteriophage and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

For expression of the nucleic acid, the expression vector(s) may be transfected into a host cell by standard techniques. The various forms of the term “transfection” are intended to encompass a wide variety of techniques commonly used for the introduction of exogenous DNA into a prokaryotic or eukaryotic host cell, such as electroporation, calcium-phosphate precipitation, DEAE-dextran transfection and the like.

Cell culture production and purification and characterization of variants can be realized by well-known methods of the prior art. For example, cell can be allow to grow and die (4 to 5 days) before supernatant collection, clarification by low-speed centrifugation and volume reduction by ultra-filtration, for example on Pellicon XL Filter (Millipore). The concentrated culture supernatants can be injected into a HiTrap protein A FF column (GE Healthcare), bound antibodies can be eluted with sodium citrate buffer, and fractions can be neutralized using Tris. Fractions containing the variants can be pooled and dialyzed into PBS, and the samples can be sterile-filtered and stored at 4° C. The purified variants can be characterized by SDS-PAGE under non-reducing and reducing conditions.

Another object of the invention relates to a method for increasing the binding affinity of a canine IgG Fc region for the canine FcRn, compared to the corresponding wild type or parent canine IgG Fc domain, said method comprising modifying a canine IgG Fc domain as mentioned above.

Another object of the invention relates to a method for increasing the in vivo half-life of a canine antibody or an Fc fusion protein, compared to the corresponding wild type or parent canine antibody or Fc fusion protein, said method comprising modifying the canine IgG Fc domain as mentioned above.

“Wild type IgG Fc region” refers to a canine IgG Fc region that does not comprise the mutation(s) described herein. It may be a canine IgG Fc region as shown in SEQ ID NO: 3, with or without the hinge region, or SEQ ID NO: 4.

Canine IgGB WT = SEQ ID NO: 3
(the hinge region is underlined)
KRENGRVPRPPDCPKCPAPEMLGGPSVFIFPPKPKDTLLIART
PEVTCVVVDLDPEDPEVQISWFVDGKQMQTAKTQPREEQFNGT
YRVVSVLPIGHQDWLKGKQFTCKVNNKALPSPIERTISKARGQ
AHQPSVYVLPPSREELSKNTVSLTCLIKDFFPPDIDVEWQSNG
QQEPESKYRTTPPQLDEDGSYFLYSKLSVDKSRWQRGDTFICA
VMHEALHNHYTQESLSHSPGK
Canine IgGD WT = SEQ ID NO: 4
PKESTCKCISPCPVPESLGGPSVFIFPPKPKDILRITRTPEIT
CVVLDLGREDPEVQISWFVDGKEVHTAKTQPREQQFNSTYRVV
SVLPIEHQDWLTGKEFKCRVNHIGLPSPIERTISKARGQAHQP
SVYVLPPSPKELSSSDTVTLTCLIKDFFPPEIDVEWQSNGQPE
PESKYHTTAPQLDEDGSYFLYSKLSVDKSRWQQGDTFTCAVMH
EALQNHYTDLSLSHSPGK

where residues at position 110 in CH3 could be either an alanine (A) or a glutamine (Q) and at position 119 in CH3 could be either a glutamic acid (E) or a lysine (K). Also the C-terminal lysine can be indifferently present or absent.

Amino acid modifications allowing obtaining the canine IgG Fc domain of the invention can be made by any method known in the art and many such methods are well known and routine for the skilled artisan. For example, but not by way of limitation, amino acid substitutions, deletions and insertions may be accomplished using any well-known PCR-based technique. Amino acid substitutions may be made by site-directed mutagenesis (see, for example, Zoller and Smith: “Oligonucleotide-directed mutagenesis using M13-derived vectors: an efficient and general procedure for the production of point mutations in any fragment of DNA”, Nucleic Acids Res. 1982 Oct. 25; 10(20):6487-500 ([8])). Mutagenesis may be performed in accordance with any of the techniques known in the art including, but not limited to, synthesizing an oligonucleotide having one or more modifications within the sequence of the constant domain of an antibody or a fragment thereof (e. g., the CH2 or CH3 domain) to be modified. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered. A number of such primers introducing a variety of different mutations at one or more positions may be used to generate a library of mutants. The technique of site-specific mutagenesis is well known in the art, as exemplified by various publications (see, e. g.,. Kunkel et al.: “Rapid and efficient site-specific mutagenesis without phenotypic selection”, Methods Enzymol. 1987; 154:367-82 ([9])). In general, site-directed mutagenesis is performed by first obtaining a single-stranded vector or melting apart of two strands of a double stranded vector which includes within its sequence a DNA sequence which encodes the desired peptide. An oligonucleotide primer bearing the desired mutated sequence is prepared, generally synthetically. This primer is then annealed with the single-stranded vector, and subjected to DNA polymerizing enzymes such as T7 DNA polymerase, in order to complete the synthesis of the mutation-bearing strand. Thus, a heteroduplex is formed wherein one strand encodes the original non-mutated sequence and the second strand bears the desired mutation. This heteroduplex vector is then used to transform or transfect appropriate cells, such as E. coli cells, and clones are selected which include recombinant vectors bearing the mutated sequence arrangement. As will be appreciated, the technique typically employs a phage vector which exists in both a single stranded and double stranded form. Typical vectors useful in site-directed mutagenesis include vectors such as the M13 phage. These phage are readily commercially available and their use is generally well known to those skilled in the art. Double stranded plasm ids are also routinely employed in site directed mutagenesis which eliminates the step of transferring the gene of interest from a plasmid to a phage.

Alternatively, the use of PCR™ with commercially available thermostable enzymes such as Taq DNA polymerase may be used to incorporate a mutagenic oligonucleotide primer into an amplified DNA fragment that can then be cloned into an appropriate cloning or expression vector. PCR™ employing a thermostable ligase in addition to a thermostable polymerase may also be used to incorporate a phosphorylated mutagenic oligonucleotide into an amplified DNA fragment that may then be cloned into an appropriate cloning or expression.

Mutants that result in increased affinity for FcRn and increased in vivo half-life may readily be screened using well-known and routine assays. In a preferred method, amino acid substitutions are introduced at one or more residues in the IgG constant domain or FcRn-binding fragment thereof and the mutated constant domains or fragments are expressed on the surface of bacteriophage which are then screened for increased FcRn binding affinity.

Another object of the invention relates to a composition, preferably a pharmaceutical composition, comprising a canine IgG Fc domain of the invention, or an antibody of the invention, or an Fc-fusion protein of the invention.

Another object of the invention relates to a canine IgG Fc domain of the invention, or an antibody of the invention, or an Fc-fusion protein of the invention, for use as a drug.

Another object of the invention relates to a canine IgG Fc domain of the invention, or an antibody of the invention, or an Fc-fusion protein of the invention, for use in the treatment of a disease selected among an inflammatory disease, an auto-immune disorder, an IgG mediated autoimmune disease, an immune-mediated disease, osteoarthritis, atopic dermatitis, skin inflammatory disease, otitis, infectious disease, and respiratory disease.

Another object of the invention relates to an IgG Fc domain of the invention, or an antibody of the invention, or an Fc-fusion protein of the invention, for use in the treatment of infectious or parasitic diseases.

Another object of the invention relates to the use of an IgG Fc domain of the invention, or of an antibody of the invention, or of an Fc-fusion protein of the invention, as a diagnostic tool or as a research tool, especially research of inflammatory or immune disease treatments, or for zootechnical applications.

As mentioned above, the antibody and the Fc-fusion protein of the invention may be therapeutic, diagnostic or for research.

In an embodiment, it may be for the research of treatment of inflammatory or immune disease. The inflammatory disease may be for example a skin inflammatory disease such as atopic dermatitis, osteo-joint disease such as osteoarthritis, cancer or immune disease such as allergy.

The antibody or the Fc-fusion protein of the invention may bind to any epitope likely to be a target for an antibody or an Fc fusion protein. It maybe:
an epitope selected among IL-31, IL31R, IL13, IL4, IL4R, IL13R, IL-5, IL23, IL22, IGF-1, CCL17, CD14, CD20, CD52 , CD40, CD50, CD80, CD154, CD163, CX3CL1, CCR2, CXCR2, CGRP, CHST14 antibodies, TNF-α, TNFR1 HER-1, HER-2, Ig-E, NGF, PD-1, PD-L1, Nav1.3, Nav1.5, Nav1.7, TSLP, TGF-β, p53 protein, Flt3 ligand, GM-CSF, protein or peptide of myelin oligodendrocytes glycoprotein, MMP-13, MMP-3, MMP-1, ADAMTS-4, ADAMTS-5, uPA, uPAR, soluble receptor involved in blockade activation or modulation of innate immunity or adaptive immunity related to inflammatory disease, for example TSLPR or TARC, 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAM9, ADAMTS, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CA125, CAD-8, Calcitonin, cAMP, carcinoembryonic antigen (CEA), carcinoma-associated antigen, Cathepsin A, Cathepsin B, Cathepsin C/DPPI,

Cathepsin D, Cathepsin E, Cathepsin H, Cathepsin L, Cathepsin O, Cathepsin S, Cathepsin V, Cathepsin X/ZIP, CBL, CCI, CCK2, CCL, CCL1, CCL11, CCL12, CCL13, CCL14, CCL15, CCL16, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD15, CD16, CD18, CD19, CD21, CD22, CD23, CD25, CD27L, CD28, CD29, CD30, CD30L, CD32, CD33 (p67 proteins), CD34, CD38, CD40L, CD44, CD45, CD46, CD49a, CD54, CD55, CD56, CD61, CD64, CD66e, CD74, CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, EGAD, EDA, EDA-A1, EDA-A2, EDAR, EGF, EGFR (ErbB-1), EMA, EMMPRIN, ENA, endothelin receptor, Enkephalinase, eNOS, Eot, eotaxin1, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MM), HIV gp120, HIV IIIB gp120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IGF, IGF binding proteins, IGF-1R, IGFBP, IGF-I, IGF-II, IL, IL-1, IL-1R, IL-2, IL-2R, IL-5, IL-5R, IL-6, IL-6R, IL-8, IL-9, IL-10, IL-12, IL-15, IL-18, IL-18R, interferon (INF)-alpha, INF-beta, INF-gamma, Inhibin, iNOS, Insulin A-chain, Insulin B-chain, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bp1, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibitin substance, Mug, MuSK, NAIP, NAP, NCAD, N-Cadherin, NCA 90, NCAM, NCAM, Neprilysin, Neurotrophin-3, -4, or -6, Neurturin, NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, 5100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha beta, TNF-beta2, TNFc, TNF-RII, TNFRSF10A (TRAIL R1 Apo-2, DR4), TNFRSF10B (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF RI CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, tumor-associated antigen CA 125, tumor-associated antigen expressing Lewis Y related carbohydrate, TWEAK, TXB2, Ung, uPAR-1, Urokinase, VCAM, VCAM-1, VECAD, VE-Cadherin, VE-cadherin-2, VEFGR-1 (flt-1), VEGF, VEGFR, VEGFR-3 (fit-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and receptors for hormones and growth factors, or
a parasite epitope selected in the group comprising an exposed antigen including T-cell activation inhibitors for example p36, Iris, Salp15 and IL-2 binding protein, 64P tick protein, Salp15, Salp25D, HL34, P29, RIM36, caltericulin, Tick Histamine Release Factor (tHRF) and AamAV422, a concealed antigen for example Bm86 protein, ferritins for example ferritin 2, HIFER1 and HIFER2, serpins (Serine protease inhibitors) for example RAS-3, RAS-4 and RIM36, 4D8, Subolesin (SUB)/Akirin, HLS1 serine proteinase inhibitor, HLS2 serine proteinase inhibitor, P27/P30 troponin I-like protein, caltericulin, Bm91, voraxin, peritrophin, akirins, midgut mucins, Manα1-6 proximal to Galβ1-4GlcNAc-α-O-R glycans, Maxadilan factor (MAX), Salivary Gland Lysate (SGL), Salivary Gland Protein 15 (SP15), microfilarial IgM-activating antigens for example polypeptide P34, polypeptide P38, 14-16 kDa microfilarial antigens, 63 kDa microfilarial antigen or 73 kDA microfilarial antigen, microfilarial IgG-activating antigens for example 36kDa antigen, 38 kDa antigen, 71 kDa antigen or 84 kDa antigen, third stage larval antigens for example polypeptides P200, P130, P100, P80, P75, P38, P34, P32, P21, P15, 14 kDa antigen, 20 kDa antigen, 30 kDa antigen, 34 kDa antigen, 35 kDa major surface antigen or 39 kDa antigen, fourth stage larval antigens for example 39 kDa antigen, 66 kDa antigen, 24/23 kDa doublet antigen, 15 kDa antigen, 31 kDa antigen, 39 kDa antigen, 42 kDa antigen, 55 kDa antigen, 59 kDa antigen, 70 kDa antigen, 97 kDa antigen or 207 kDa antigen, adult stage antigens for example 15 kDa antigen, 20 kDa antigen or 38 kDa antigen, and a universal antigen for example DiAg, Di5 (cuticular) antigen, somatic antigens for example tropomyosin, major sperm protein, P22U or small heat shock protein 12.6, a surface antigen for example papain-like cysteine proteinase or GADPH (Glyceraldehyde 3-phosphate dehydrogenase), Excretory-Secretory (E/S) products for example Triose Phosphate Isomerase, Heat Shock Protein 70 (HSP70) and Transthyretin, or
a pathogen or pathogen-derived material for example lipopolysaccharides, peptidoglycans, cell wall components, cell membrane components, toxins, siderophores, virulence factors, adhesins or molecules involved in quorum sensing, receptors involved in activation, blockade or modulation of innate immunity or adaptive immunity, for example Pattern Recognition Receptors like Toll-like receptors or C-type Lectin Receptors, G-Protein Coupled Receptors, costimulatory membrane proteins or immune checkpoint inhibitors such as B7 protein family, Programmed cell Death molecules (PD) and PD ligands (PD-L), CTLA-4, or LAG-3, and cytokines for example chemokines, interleukins, interferons, mediators involved in promotion or resolution of inflammation, in activation, blockade or modulation of innate immunity or adaptive immunity.

As mentioned above, the canine IgG Fc domain or the antibody, or the Fc-fusion protein of the invention may be for use as a drug, which may be a therapeutic or prophylactic drug. For example, it may be a vaccine.

The drug may allow delivery of protein, for example antibody, hormones or growth factor, across epithelial barrier, as mammary gland epithelium, intestinal, pulmonary, vaginal or other mucosal barrier.

As mentioned above, the canine IgG Fc domain, or the antibody, or the Fc-fusion protein of the invention may be for use in the treatment of an auto-immune disorder, for example an IgG mediated autoimmune disease, or an autoimmune disease selected among bullous autoimmune skin disease, systemic lupus erythematosus, autoimmune hemolytic anemia, immune-mediated thrombocytopenia, thrombocytopenia, autoimmune blood disease, autoimmune musculoskeletal system disease, autoimmune thyroid disease, multiple organ autoimmune diseases, autoimmune adrenal gland autoimmune disease and hypothyroidism. The bullous autoimmune skin disease may include pemphigus vulgaris, pemphigus foliaceus, pemphigus vegetans, pemphigus erythematosus and bullous pemphigoid thyroid. According to the invention, the autoimmune blood disease may be selected among autoimmune haemolytic anaemia, immune-mediated thrombocytopenia and systemic lupus erythematosus. According to the invention, the autoimmune musculoskeletal system disease is selected among myasthenia gravis, rheumatoid arthritis, systemic lupus erythematosus and polyarthritis. In an embodiment of the invention, the autoimmune thyroid disease may be associated with lymphocytic thyroiditis. The multiple organ autoimmune disease may be selected among systemic lupus erythematosus and discoid lupus erythematosus. The autoimmune adrenal gland autoimmune disease may be for example hypoadrenocorticism.

As mentioned above, the canine IgG Fc domain, or the antibody, or the Fc-fusion protein of the invention may be for use in the treatment of an inflammatory disease, which may be a skin inflammatory disease such as atopic dermatitis, or an osteo-joint disease such as arthrosis, cancer or allergy.

As mentioned above, the canine IgG Fc domain, or the antibody, or the Fc-fusion protein of the invention may be for use in the treatment of of infectious or parasitic diseases, which may be selected among diseases induced by ectoparasites and endoparasites of dogs, namely ticks (Arachnida: Ixodida), mites (Arachnida: Acari), chewing and biting lice (Arthropoda: Phthiraptera), fleas (Arthropoda: Siphonaptera), flies (Diptera: Nematocera and Brachycera), mosquitoes (Diptera: Culicidae), sand flies (Diptera: Psychodidae), nematodes (Nemathelminthes: Nematoda), trematodes (Plathelminthes: Trematoda), cestodes (Plathelminthes: Cestoda) and protozoa (Protista: Protozoa) and respiratory infections, urinary infections and dermatological infections, notably skin infections, soft tissues infections and otitis.

As mentioned above, the canine IgG Fc domain, or the antibody, or the Fc-fusion protein of the invention may be for use in the treatment of infectious respiratory infections, which may be selected among diseases induced by Bordetella bronchiseptica, Mycoplasma spp (M. canis, M.cynos), Streptococcus spp, Escherichia coli, Pasteurella multocida, Staphylococcus spp, CIV/Canine influenza virus, CPIV/canine parainfluenza virus, CnPnV/Canine pneumovirus, CDV/Canin distemper virus, CRCoV/Canine respiratory coronavirus, CAdV-2/Canine adenovirus type 2, and CaHV-1/Canine herpesvirus type 1. The urinary infections may be selected among diseases induced by Staphylococcus pseudintermedius, Staphylococcus aureus, Coagulase-negative staphylococcus spp, Pseudomonas aeruginosa, Proteus spp, Escherichia coli, Corynebacterium spp, Enterococcus spp, Citrobacter spp, Enterobacter spp, Mycoplasma spp, Lactobacillus spp, Klebsiella spp, and Anaerobic bacteria.

The skin and soft tissues infections may be selected among diseases induced by Staphylococcus pseudintermedius, Staphylococcus aureus, Pseudomonas aeruginosa, Proteus spp, Escherichia coli, Corynebacterium spp, Enterococcus spp, Citrobacter spp, Lactobacillus spp, Klebsiella spp, Anaerobic bacteria, Malassezia pachydermatis, and Malassezia spp.

As mentioned above, the IgG Fc domain, or the antibody, or the Fc-fusion protein of the invention, may be used as a diagnostic tool or as a research tool for zootechnical applications, which may be selected among control of reproduction of animals, as animal's oestrus cycle and castration. A further object of the invention is to provide pharmaceutical compositions comprising the said variant. The said formulations are prepared by mixing the polypeptide variant having the desired degree of purity with optional physiologically acceptable pharmaceutically acceptable carrier, excipients or stabilizers in the form of lyophilised formulations or aqueous solutions. Such pharmaceutical compositions are destined for treating a patient in need.

In order to treat a patient in need, a therapeutically effective dose of the variant may be administered. By “therapeutically effective dose” herein is meant a dose that produces the effects for which it is administered. The exact dose will depend on the purpose of the treatment, and will be ascertainable by one skilled in the art using known techniques. Dosages may range from 0.001 to 100 mg/kg of body weight or greater, for example 0.1, 1.0, 10, or 50 mg/kg of body weight, with 0.1 to 10mg/kg being preferred. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

Administration of the pharmaceutical composition comprising a variant may be done in a variety of ways, including, but not limited to, orally, subcutaneously, intravenously, parenterally, intranasally, intraortically, intraocularly, rectally, vaginally, transdermal, topically (e.g., gels, salves, lotions, creams, etc.), intraperitoneal, intramuscularly, intrapulmonary.

Therapeutic described herein may be administered with other therapeutics concomitantly, i.e., the therapeutics described herein may be co-administered with other therapies or therapeutics, including for example, small molecules, other biologicals, radiation therapy or surgery, this list not being limitative.

This invention is further illustrated by the following examples with regard to the annexed drawings that should not be construed as limiting.

### BRIEF DESCRIPTION OF THE DRAWINGS

FIG. 1 represents the comparative binding analysis (RU/s) of YTE-b12 canine IgGB (YTE) vs. wild-type b12 canine IgGB (WT), with WT (920 nM and 460 nM) at pH 6.0 (triangles), WT (920 nM and 460 nM) at pH 7.4 (circles), YTE (920 nM and 460 nM) at pH 6.0 (squarres), YTE (920 nM and 460 nM) at pH 7.4 (diamond).

FIG. 2 represents the comparative binding analysis (RU/s) of NA-b12 canine IgGB (NA) vs. wild-type b12 canine IgGB (WT), with WT (920 nM and 460 nM) at pH 6.0 (squarres), WT (920 nM and 460 nM) at pH 7.4 (circle), NA (920 nM and 460 nM) at pH 6.0 (diamonds), and NA (920 nM and 460 nM) at pH 7.4 (triangle).

FIG. 3 represents the comparative binding analysis (RU/s) of AAA b12 canine IgGB (AAA) vs. wild-type b12 canine IgGB (WT) Fc, with WT (920 nM and 460 nM) at pH 6.0 (squarres), WT (920 nM and 460 nM) at pH 7.4 (circle), AAA (920 nM and 460 nM) at pH 6.0 (diamonds), and AAA (920 nM and 460 nM) at pH 7.4 (triangle).

FIG. 4 represents mean Ig plasma concentration (μg/mL)-time (h) profiles following a single intravenous administration in dogs at a dose of 0.2 mg/kg, semi-logarithmic scale for variant YTE (circles) and wild type (triangle).

FIG. 5: represents the asymmetric unit of the Fc, FcRn, HSA complex from the structure of PDB 1D 4NOU. The HSA domain is located at the far lower right of the figure, the FCGRT domain of FcRn is located at the lower center of the figure, the B2M domain of FcRn is in located at the upper center of the figure, and the Fc domain including with its carbohydrate adduct is in located at the far left of the figure. Molecular images generated in Pymol ([12]).

FIG. 6: represents the symmetry-expanded Fc/FcRn complex, identified as in FIG. 8, with the dimeric Fc domain in the center and the carbohydrate adduct removed (Molecular images generated in Pymol ([12])).

FIG. 7: represents a stereo view of the locations of the M15A, S16 and T18 residues (IMGT numbering, shown as sticks) at the Fc CH2-CH3 junction (Molecular images generated in Pymol ([12])).

FIG. 8: represents a multiple sequence alignment encompassing the IgG sequences modeled in this study. For reference, the sections of the canine sequence that are mutually identical are boxed, the sections of the human sequence that are identical between human and canine are boxed on the human sequence, and the sections of the feline sequence that are identical between all three species are boxed on the feline sequence. The residues identified as belonging to the binding interface of the native Fc/FcRn complex (based upon the ca-IgGD/FcRn) are shaded. Finally, the locations of the seven residues involved in the five mutation groups are marked with stars.

FIG. 9: represents a stereo view of the environment of the ca-IgGD-YTE mutation group, R(E15A)Y; T(E16)T; T(E18)E (Molecular images generated in Pymol ([12]))

FIG. 10: represents a stereo view of the environment of the fe-IgG1A YTE mutation group, S(E15A)Y; S(E16)T, T(E18)E (Molecular images generated in Pymol ([12])).

### EXAMPLES

### Example 1: Preparation of Mutated Canine IgG Fc Domain

### Gene Synthesis

The amino acid sequences corresponding to the variable regions of B12 heavy chain (VH) and light chain (VL), an anti-HIV-1 gp120 neutralizing antibody (Zhou T et al.: “Structural definition of a conserved neutralization epitope on HIV-1 gp120” Nature. 2007 Feb. 15; 445(7129):732-7 ([10])) were used to design the DNA sequences after codon optimization for mammalian expression. For the heavy and light chains, the DNAs encoding a unique Notl restriction site followed by a consensus Kozak sequence (GCCGCCACC) followed by signal peptide (MGVPTQLGLLLWLTDARC (SEQ ID NO:5) for the light chain and MEWSWVFLFFLSVTTGVHS (SEQ ID NO:6) for the heavy chain), the B12 variable regions (VH and VL) and a unique restriction site (Nhel for VH and BsiWI for VL) were synthesized. The VH and VL constructs delivered in shuttle vectors were digested by restriction enzymes (Not I and Nhel for VH and NotI and BsiWI for VL) and ligated in pcDNA3.1 expression vector (Invitrogen) in which the canine IgG2 (isotype B=chain B) CH1+hinge+CH2+CH3 domains were already inserted for the VH and pCDNA3.1 expression vector in which the canine Kappa constant domain was already inserted for the VL. Plasmid DNAs were verified by double strand DNA sequencing.

Variants of the parental B12-canine IgG2 molecule were obtained by introducing mutations in the Fc regions (CH2 and/or CH3 domains) using QuikChange Site-Directed mutagenesis kit from Stratagene.

Expression and Purification of 812-canine IgG2 and Fc Variants

Recombinant monoclonal antibodies were produced by means of transient gene expression by co-transfection of 2 genes coded on separate vectors in CHO-S cells adapted to serum-free medium in suspension (CHO SFM-II medium from Life TechnologiesTM). Typically, for 50 mL medium scale expression testing, a total of 50 μg of plasmid DNAs (25 pg heavy chain and 25 μg light chain were mixed in 1.5 mL Eppendorf tube, 1 mL of CHO SFM medium containing 25 μL of 3 mg/mL PEI transfection reagent (Polyplus) pH 7.0 was added, incubated at RT for 20 min. The mixture of DNA-PEI was loaded into 49 mL of Life Technologies' Invitrogen FreeStyle™ CHO-S cells at 1-2×10⁶/mL in 125 mL shaking flask. Cells were shaken for 6 more days. The supernatant was harvested by centrifuging cells at 3,000 rpm for 15 min. The expression titer of the canine IgG in the supernatant was determined using FortéBio's protein A biosensors (Octet® Systems). The parental B12 canine IgG2 monoclonal antibody and Fc-variants were then purified on protein A affinity medium using MabSelect SuRe (GE Healthcare Life Sciences). The antibodies were eluted from protein A using 0.1 M glycine pH 3.5 with neutralization in 1 M

TRIS. The purified antibodies in Dulbecco's PBS (Lonza BE17-512Q) were sterile-filtered (0.2 μM sterile filters from Techno Plastic Products AG) and the final concentration determined by OD reading at 280 nm using Eppendorf BioSpectrometer®.

### Canine FcRn-β2M

Canine FcRn with an enzymatically biotinylated C-terminus and produced with the corresponding canine beta 2 macroglobulin was purchased from Immunitrack (Denmark, # ITF11-1000).

### SPR Analysis

SPR experiments are carried out at 25° C. using a BlAcore 2000 (Biacore AB, Uppsala, Sweden). A CM5 chip is coated with streptavidin (Roche, Basel, Switzerland) by amine coupling. Unused activated chip surface is blocked injecting 1 M ethanolamine. The BlAcore is primed with HBS-EP buffer (Biacore AB, Uppsala, Sweden) at pH 8.0. Biotinylated canine FcRn-β2M complex diluted in HBS-EP buffer at pH 8.0 is immobilized on the streptavidin sensor surface. The flow cell 1 coated with streptavidin is used as a reference cell. For kinetic experiments, B12 canine IgG2 parental molecule and Fc-variants are injected in HBS-EP buffer pH 6.0. At the end of the dissociation phase, HBS-EP buffer pH 8.0 is injected for surface regeneration. Each injection is carried out in duplicate. To avoid nonspecific binding and bulk buffer effects, data are processed by subtracting signals obtained from the control surface and the blank injection using the BlAevaluation software. Affinity constants are determined according to the bivalent analyte model.

### Pharmacokinetic Study in Dogs After Single Intravenous Injection of 812-Canine IgG2 and Fc Vvariants

Male adult beagle dogs (approximately 8-10 kg, n=3 per group) receive a single intravenous bolus injection of the parental B12 canine IgG2 mAb or an Fc-variant at a dose of 0.2 mg/kg. In time intervals of 5 min, 30 min, 1 h, 4 h, 7 h, 1 day, 2 days, 3 days, 7 days, 14 days and 28 days following IV administration, blood samples are taken from the peripheral vein using vacuum tubes containing anticoagulant agent. Blood samples are incubated on ice. Clotted blood is centrifuged at 13,000 g for 30 min at 4° C., and serum samples are stored at −20° C. Plasma canine IgG concentrations are determined by ELISA assay (as described below). The pharmacokinetic parameters, half-lives (t1/2α, t1/2β) and AUC, are calculated according to standard non-compartmental methods, using Phoenix WinNonlin software.

### ELISA Method for Quantitation of B12-IgG2 mAb Concentration in Male Beagle Dog Serum Samples

Recombinant HIV1 gp120 protein (ab73769 from Abcam) is immobilized on ELISA plates overnight at a concentration of 1 μg/ml at 4° C. and the remaining binding sites are blocked with 2% (w/v) non-fat dry milk/phosphate buffered saline (MPBS). Purified recombinant b12-canine IgG2, Fc variants or serum samples are diluted in MPBS, titrated in duplicates and incubated for 1 h at room temperature. Detection is performed with horse radish peroxidase (HRP) conjugated secondary antibody (anti-canine IgG antibody) using 100 μl 3,3′,5,5′-tetramethylbenzidine (TMB) substrate (0.1 mg/ml TMB, 100 mM sodium acetate buffer pH 6, 0.006% H2O2). The reaction is stopped with 50 μl of 1 M H₂SO₄ and absorbance is measured at 450 nm. Data are fitted with GraphPrism software (La Jolla, Calif., USA) and the concentration of recombinant canine IgG in the serum calculated.

### Example 2: Measure of In Vitro Canine mAbs-FcRn Interactions

The canine IgG Fc mutant YTE, comprising the substitutions (IMGT numbering system) of amino acid 15.1 of CH2 domain with tyrosine, of amino acid 16 of CH2 domain with threonine, and of amino acid 18 of CH2 domain with glutamic acid (“YTE variant”), is prepared as in example 1 above.

Also, mutant N114A, which corresponds to a canine IgG Fc domain, the amino acid sequence of which comprising the substitution of amino acid 114 of CH3 domain with alanine (“NA” mutation), and mutant N90A N40A N114A which corresponds to a canine IgG Fc domain, the amino acid sequence of which comprising the substitution of amino acid 90 of CH2 domain with alanine, the substitution of amino acid 40 of CH3 domain with alanine and the substitution of amino acid 114 of CH3 domain with alanine (“AAA” mutation), are prepared as in Example 1.

Immobilisation of biotinylated FcRn to C1 Chip to measure the interactions between canine IgG2 (B) and the canine FCGRT/B2M (FcRn) complex was used in the first instance. Streptavidin 50 μg/ml in 10mM Acetate Buffer pH 4.5 was used to functionalize a C1 Chip (ca 180RU). FcRn-biotinylated was applied at 0.5, 1.5 and 10 ng/ml to prepare a low density surface for interaction analysis at Rmax between 15-30RU. Rmax was tested using canine IgG at 50 μg/ml.

We observed a high non-specific background to the chip at pH 6.0 which means that a different experiment set up had to be used. The problem has been observed on different surfaces, so in the second instance we turned the setup around, and capture antibody to inject FcRn as a soluble ligand.

A protein L surface was prepared and tested. This surface does nicely bind the b12 canine IgG2 (B) and allows to look at the interaction without severe background problems.

The comparative Binding Analysis was performed with the following parameters:
Setup: Fc2-1 Protein L,
Buffers 20mM Phosphate 150 mM NaCl pH 6.0 and 7.4
Temperature 25° C.
Capture 1200RU antibody mutant
Ligand 920 and 460 nM FcRn in respective buffer.

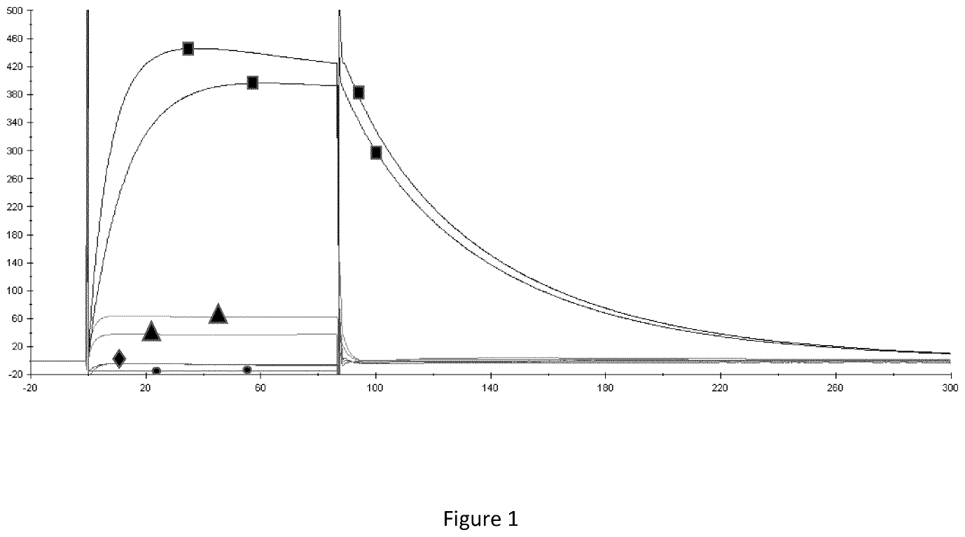

At pH 6.0, the YTE mutant exhibited strong binding as compared to that of WT. A Kd value of 150 nM could be generated for the YTE mutant. At pH 7.4, the YTE mutant showed minimum residual binding See FIG. 1.

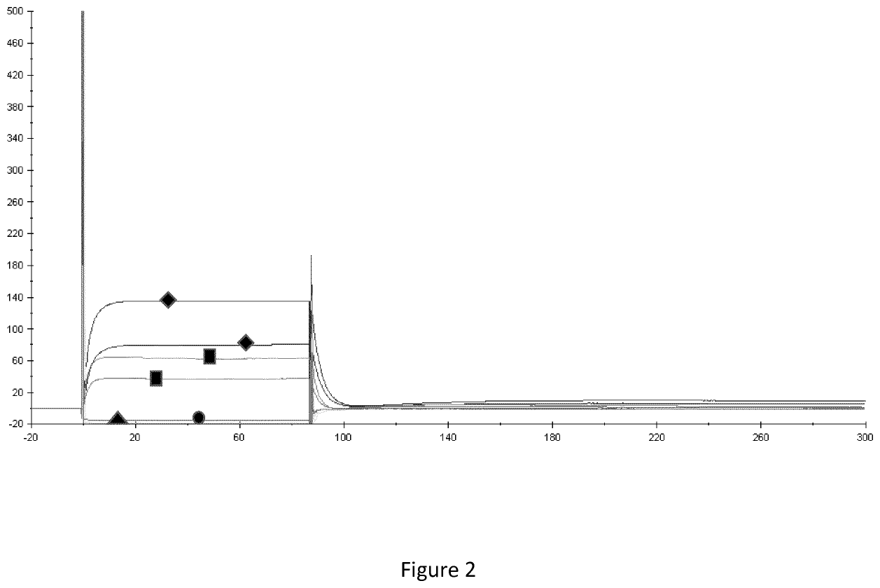

At pH 6.0 the NA mutant has a slightly better binding than WT (by higher on-rate) but no near as strong as YTE mutant. No Kd value could be calculated. A pH 7.4, there is no residual binding to FcRn. See FIG. 2

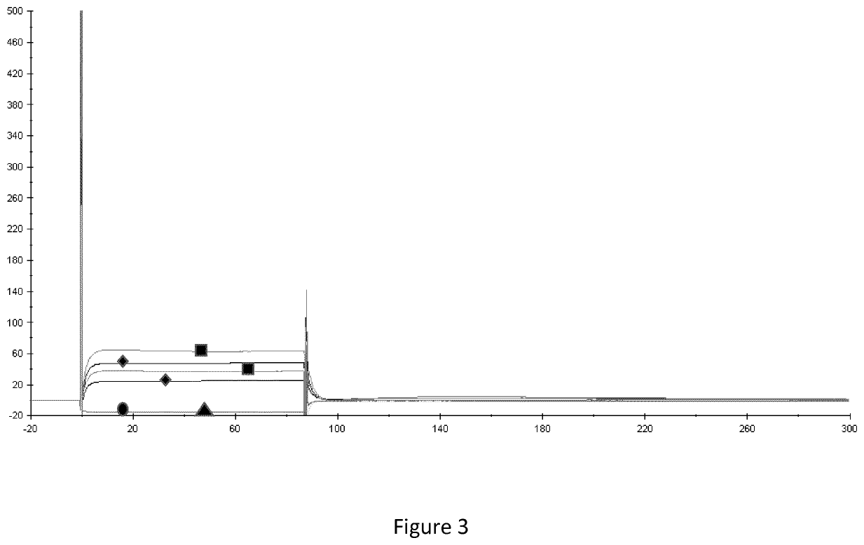

At pH 6.0; the AAA mutant has a lower binding than the WT. A pH 7.4, there is no residual binding to FcRn. See FIG. 3.

### Example 3: Determination of the Pharmacokinetic Parameters of Canine WT b12-IgGB and Canine YTE-b12-igGB Variant in Dogs After Intravenous Administration at a Dose of 0.2 mg/kg

The objective of this study is to verify that the elimination half-life time of an immunoglobulin (Ig) containing a modified Fc fragment is greater than that of the wild-type Ig in the canine species.

To do so, the plasma pharmacokinetics of these 2 canine IgG in dogs are compared after intravenous administration at a dose of 0.2 mg/kg:

Immunoglobuline A: wild type

Immunoglobuline B: YTE variant

### Animals

Nine male and/or female beagle dogs weighing between 8.0 kg and 17.5 kg at baseline participate to the study. The breed, weight, sex, date of birth and origin of the animals are listed in the Table 1 below:

TABLE 1
Characteristics of the animals used in the study

| | | | | | Age (years) at |
|---|---|---|---|---|---|
| Dog’s | | | | | the time of |
| study | | | Bodyweight | Date of | administration |
| number | Origin | Sex | (kg) | birth | (on 9 May 2017) |
| 1 | CEDS | F | 14.9 | 21 Aug 2014 | 2.7 |
| 2 | CEDS | F | 14.1 | 21 Aug 2014 | 2.7 |
| 3 | Isoquimen | F | 14.7 | 15 Oct 20213 | 3.6 |
| 4 | CEDS | F | 15.3 | 24 Aug 2015 | 1.7 |
| 5 | CEDS | F | 13.5 | 24 Aug 2015 | 1.7 |
| 6 | CEDS | F | 14.3 | 22 Aug 2015 | 1.7 |

### Conditions of the Assays

Immunoglobulins are used as injectable solutions at 2 mg/mL.

Immunoglobulin solutions should be packed in 4 mL tubes. The solutions should be stored at −20° C. in their original packaging and returned to room temperature before injection. In case of precipitation after thawing, the solutions are vortexed. If the precipitate is still present after vortexing, the tube is not used for administrations.

### Experimental Design of the Study

Intravenous administration is carried out on all animals on Day 0 (DO). The animals are divided into 2 groups of 3 animals, as shown in Table 2 below.

TABLE 2
Actual administered doses by intravenous route

| | | |
|---|---|---|
| Group 1: dogs 1 à 3 | Administration | Immunoglobulin A: wild type |
| Group 2: dogs 4 à 6 | (D0) | Immunoglobulin B: YTE variant |

For the calculation of the volumes to be administered, the dogs are weighed during a clinical examination at Day 4.

### Administration of the Solutions

Injectable immunoglobulin solutions are administered intravenously (IV, slow bolus) to the cephalic vein at a dose of 0.2 mg/kg, corresponding to a volume of 0.1 mL/kg. Immunoglobulin solutions are packaged in 4 mL tubes.

An anti-reflux catheter is used. The catheter is rinsed with 1 mL of physiological saline (0.9% NaCl) immediately after injection of the immunoglobulin solution. The catheter is left in place for at least 2 hours after administration, in order to inject shock treatment drugs if necessary.

TABLE 3
Actual administered doses by intravenous route

| Dog | | | | |
|---|---|---|---|---|
| study | Bodyweight | | Administered | Administered |
| number | (kg) | Ig | volume (mL) | dose (mg/kg) |
| 1 | 14.9 | Wild type | 1.5 | 0.20 |
| 2 | 14.1 | | 1.4 | 0.20 |
| 3 | 14.7 | | 1.5 | 0.20 |
| 4 | 15.3 | Variant YTE | 1.5 | 0.20 |
| 5 | 13.5 | | 1.4 | 0.21 |
| 6 | 14.3 | | 1.4 | 0.20 |

### Blood Samples

The blood (approximately 4 mL) are removed by direct puncture of the jugular vein in a heparinized tube (Lithium Heparin). Blood samples may be taken from fasted or unfasted animals.

The sampling time before administration (T0=pre-dose) is realized at D0.

Immediately after sampling, the blood tubes are protected from light on a rack placed on a bed of ice (the tubes do not come into contact with the ice).

The blood tubes are transmitted within two hours to the bioanalysis laboratory where they are centrifuged (the centrifuge is set to 3500 rpm) for 10 minutes at about 5° C. The plasma TO (pre-dose) is divided into 8 aliquots of 150 μL minimum. The plasma for the remaining sampling times is divided into 3 aliquots of 400 μL minimum. Plasma aliquots are placed in a freezer at the bioanalysis laboratory at about −75° C. until assayed.

TABLE 4
Actual blood sampling times
after intravenous administration

| Theo- | | | | | | |
|---|---|---|---|---|---|---|
| retical | Ig Wild type | | | Ig Variant YTE | | |
| time (h) | Dog 1 | Dog 2 | Dog 3 | Dog 4 | Dog 5 | Dog 6 |
| 0.08 | 0.08 | 0.08 | 0.08 | 0.08 | 0.08 | 0.08 |
| 0.5 | 0.50 | 0.50 | 0.50 | 0.52 | 0.50 | 0.50 |
| 1 | 1.00 | 1.00 | 1.00 | 1.00 | 1.00 | 1.00 |
| 3 | 3.00 | 3.00 | 3.00 | 3.00 | 3.00 | 3.00 |
| 7 | 7.00 | 7.00 | 7.00 | 7.00 | 7.00 | 7.00 |
| 24 | 24.00 | 24.00 | 24.00 | 24.00 | 24.00 | 24.00 |
| 28 | 28.00 | 28.00 | 28.00 | 28.00 | 28.00 | 28.00 |
| 31 | 31.00 | 31.00 | 31.00 | 31.00 | 31.00 | 31.00 |
| 48 | 48.00 | 48.00 | 48.00 | 48.00 | 48.00 | 48.00 |
| 55 | 55.00 | 55.00 | 55.00 | 55.00 | 55.00 | 55.00 |
| 75 | 74.75 | 74.53 | 74.38 | 74.68 | 74.48 | 74.32 |
| 168 | 167.92 | 167.70 | 167.57 | 167.87 | 167.63 | 167.50 |
| 336 | 335.83 | 335.70 | 335.47 | 335.78 | 335.50 | 335.43 |
| 507 | 503.78 | 503.57 | 503.42 | 503.73 | 503.50 | 503.38 |
| 672 | 671.78 | 671.58 | 671.42 | 671.72 | 671.53 | 671.37 |
| 840 | 839.78 | 839.60 | 839.47 | 839.73 | 839.55 | 839.40 |
| 1008 | 1007.75 | 1007.57 | 1007.43 | 1007.70 | 1007.52 | 1007.38 |
| 1176 | 1176.98 | 1175.53 | 1175.32 | 1176.18 | 1176.52 | 1175.47 |

### Measurement of Ig Seric Concentrations in Biological Specimens

The Ig are assayed in the serum samples by an ELISA method.

TABLE 5
wild type Ig plasma concentrations following a single
intravenous administration in dogs at a dose of 0.2 mg/kg

| | Theo- | | | | | |
|---|---|---|---|---|---|---|
| | retical | Ig concentration (μg/mL) | | | | |
| Ig | time (h) | Dog 1 | Dog 2 | Dog 3 | Mean* | Sd* |
| Wild | 0 | 0.000 | 0.000 | 0.000 | 0.0000 | 0.00000 |
| type | (pre- | | | | | |
| | dose) | | | | | |
| | 0.08 | 2.468 | 2.561 | 2.481 | 2.503 | 0.05036 |
| | 0.5 | 2.614 | 2.491 | 2.334 | 2.480 | 0.1403 |
| | 1 | 2.368 | 2.357 | 2.248 | 2.324 | 0.06634 |
| | 3 | 2.232 | 2.166 | 2.050 | 2.149 | 0.09214 |
| | 7 | 2.279 | 2.214 | 2.141 | 2.211 | 0.06904 |
| | 24 | 1.723 | 1.578 | 1.509 | 1.603 | 0.1092 |
| | 28 | 1.894 | 1.555 | 1.351 | 1.600 | 0.2743 |
| | 31 | 1.639 | 1.598 | 1.288 | 1.508 | 0.1919 |
| | 48 | 1.449 | 1.356 | 1.048 | 1.284 | 0.2099 |
| | 55 | 1.359 | 1.310 | 1.076 | 1.248 | 0.1512 |
| | 75 | 1.197 | 1.133 | 0.9972 | 1.109 | 0.1020 |
| | 168 | 0.8103 | 0.9036 | 0.6831 | 0.7990 | 0.1107 |
| | 336 | 0.4338 | 0.4536 | <lloq | 0.2958 | 0.2564 |
| | 507 | 0.2940 | 0.2442 | <lloq | 0.1794 | 0.1573 |
| | 672 | 0.1824 | 0.1673 | <lloq | 0.1166 | 0.1012 |
| | 840 | <lloq | 0.1237 | <lloq | 0.04123 | 0.07142 |
| | 1008 | <lloq | <llog | <lloq | na | na |
| | 1176 | no result | <llog | no result | na | na |

Sd: standard deviation;
lloq: lower limit of quantification (0.1 μg/mL);
na: not applicable.
*tailing concentrations <lloq were set to zero for mean and sd calculation.

TABLE 6
Ig Variant YTE plasma concentrations following a single
intravenous administration in dogs at a dose of 0.2 mg/kg.

| | Theo- | | | | | |
|---|---|---|---|---|---|---|
| | retical | | | | | |
| | time | Ig concentration (μg/mL) | | | | |
| Ig | (h) | Dog 4 | Dog 5 | Dog 6 | Mean* | Sd* |
| Variant | 0 | 0.000 | 0.000 | 0.000 | 0.000 | 0.000 |
| YTE | (pre- | | | | | |
| | dose) | | | | | |
| | 0.08 | 2.499 | 2.552 | 2.574 | 2.542 | 0.03855 |
| | 0.5 | 2.136 | 2.550 | 2.444 | 2.377 | 0.2151 |
| | 1 | 2.241 | 2.406 | 2.510 | 2.386 | 0.1356 |
| | 3 | 2.276 | 2.351 | 2.343 | 2.323 | 0.04119 |
| | 7 | 1.993 | 2.284 | 2.271 | 2.183 | 0.1644 |
| | 24 | 1.676 | 1.908 | 1.983 | 1.856 | 0.1601 |
| | 28 | 1.672 | 1.626 | 1.933 | 1.744 | 0.1656 |
| | 31 | 1.603 | 1.731 | 1.797 | 1.710 | 0.09864 |
| | 48 | 1.280 | 1.594 | 1.757 | 1.544 | 0.2425 |
| | 55 | 1.331 | 1.565 | 1.538 | 1.478 | 0.1280 |
| | 75 | 1.075 | 1.388 | 1.412 | 1.292 | 0.1880 |
| | 168 | 0.8503 | 1.207 | 1.251 | 1.103 | 0.2197 |
| | 336 | 0.7210 | 0.8882 | 0.9594 | 0.8562 | 0.1224 |
| | 507 | 0.5167 | 0.4389 | 0.6925 | 0.5494 | 0.1299 |
| | 672 | no | <lloq | 0.5937 | 0.2969 | 0.4198 |
| | | result | | | | |
| | 840 | no | <lloq | 0.4788 | 0.2394 | 0.3386 |
| | | result | | | | |
| | 1008 | no | <lloq | 0.3240 | 0.1620 | 0.2291 |
| | | result | | | | |
| | 1176 | no | no | <lloq | na | na |
| | | result | result | | | |

Sd: standard deviation;
lloq: lower limit of quantification (0.1 μg/mL);
na: not applicable.
*tailing concentrations <lloq were set to zero for mean and sd calculation.

### Evaluation of the Pharmacocinetics Parameters

The time evolution of the serum Ig concentrations is analyzed using the Phoenix WinNonlin® software (version 7.0). A non-compartmental method is used.

For the calculation of pharmacokinetic parameters, the following rules apply:
Actual sampling times are used.
The doses actually administered are used.
All concentrations below the quantitation limit (loq), located between T0 and the first concentration equal to or greater than the loq, are replaced by zero for the analysis.
Concentrations below the loq located at the end of the kinetics are not used in the calculations.

At a minimum, the following parameters are determined for each animal:

λz=the terminal elimination constant is estimated by linear logarithmic regression using at least 3 points in the terminal phase.
T_{1/2λ}z=the elimination half-life (t1/2) is calculated as follows:

$${T}_{1 / 2 {λ}_{z}} = \frac{ln ( 2 )}{{λ}_{z}}$$

AUCₗₐₛₜ=the area under the concentration curve up to the last quantifiable concentration observed is calculated using the linear trapezoid method.
AUC_{INF}=the area under the concentration curve extrapolated to infinity will be calculated as: [AUC_{INF}=AUCₗₐₛₜ+(Cₗₐₛₜ/λz)], where Clast is the last concentration of quantifiable cefalexin. The extrapolation percentage of AUCINF should normally not exceed 20%.
CI=the clearance is calculated as follows: [CI=Dose/AUC_{INF}].
MRTₗₐₛₜ=the mean residence time from the time of dosing to the time of the last quantifiable concentration, calculated as follows: MRTₗₐₛₜ=AUMCₗₐₛₜ/AUCₗₐₛₜ where AUMCₗₐₛₜ is the area under the moment curve from the time of dosing to the last quantifiable concentration.
MRT_{INF}=the mean residence time extrapolated to infinity, calculated as follows. MRT_{INF}=AUMC_{INF}/AUC_{INF} where AUMC_{INF} is the area under the moment curve extrapolated to infinity.

TABLE 7
Individual and mean plasma pharmacokinetic parameters following
a single intravenous administration in dogs at a dose of 0.2 mg/kg

| Ig | Dog | T_{1/2λz} (h) | Cl (mL/kg · h) | MRTₗₐₛₜ (h) | MRT_{INF} (h) |
|---|---|---|---|---|---|
| Wild | Dog 1 | 223.74 | 0.4150 | 202.33 | 299.11 |
| type | Dog 2 | 234.05 | 0.4119 | 228.37 | 310.01 |
| | Dog 3 | 171.42 | 0.5709 | 66.06 | 234.25 |
| | Mean | 209.74 | 0.4659 | 165.59 | 281.12 |
| | SD | 33.58 | 0.09092 | 87.17 | 40.96 |
| Variant | Dog 4 | 431.21 | 0.2623 | 201.01 | 590.91 |
| YTE | Dog 5 | 265.63 | 0.2963 | 192.88 | 357.54 |
| | Dog 6 | 448.77 | 0.1887 | 366.11 | 621.11 |
| | Mean | 381.87 | 0.2491 | 253.33 | 523.19 |
| | SD | 101.05 | 0.05500 | 97.75 | 144.25 |

λz: elimination rate constant calculated by linear regression of the last time points;
T_{1/2λz}: elimination half-life calculated with λz;
Cl: total clearance;
MRTₗₐₛₜ: mean residence time until the last measurable time point;
MRT_{INF}: mean residence time extrapolated to infinity;
SD: standard deviation.

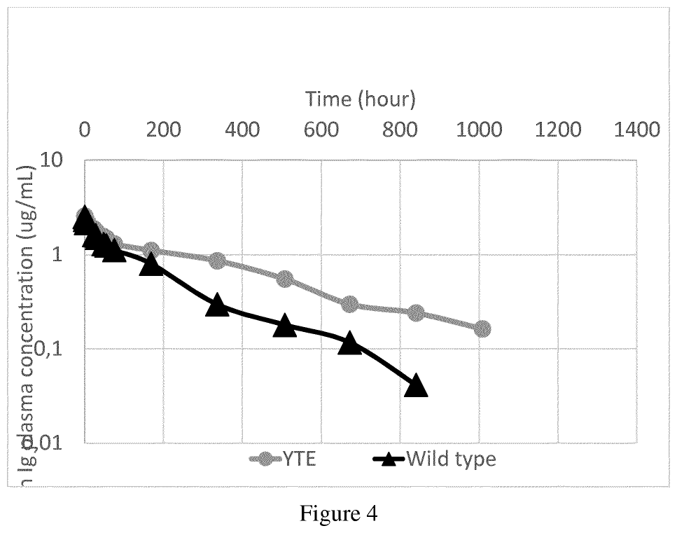

Clearance, the most useful parameter for evaluation of an elimination mechanism, is defined as the proportionality factor that relates rate of drug elimination to the plasma drug concentration. Compared to that of the wild-type antibody, Variant YTE exhibited an approximately 2-fold decrease in clearance in Beagle dogs. See FIG. 4.

The elimination half-life, T_{1/2λz}, is the time over which the plasma concentration, as well as the amount of drug in the body, falls by one half. An extended in-vivo half-life of about 1.8 fold the half-life of the wild type was observed for Variant YTE.

Another view of the events occurring following drug administration is to consider how long molecules stay in the body, before being eliminated. The average time molecules introduced reside within the body is known as the Mean Residence Time. The Mean Residence Time extrapolated to infinity, MRTINF, obtained for the variant YTE was about 1.9 times longer than that of the wild type.

### Example 4: Comparison Between Canine and Feline mAbs-FcRn Interactions

In order to compare the binding of Fc domain of Canis lupus familiaris and Felis catus to their respective neonatal Fc receptor (FcRn; FCGRT/B2M complex), molecular modeling of the complex of Fc domain of representative IgG isotype members of these species with their cognate FcRn is performed. Thus homology models of the canine IgG ca-IgG1, ca-IgG2 and ca-IgG4 Fc/FcRn complexes and that of the feline IgG1 Fc/FcRn complex have been generated based upon the 3.8 A resolution crystal structure of the YTE human Fc variant in complex with human FcRn and human serum albumin (PDB ID 4NOU) (Oganesyan V et al.: “Structural insights into neonatal fc receptor-based recycling mechanisms”, J Biol Chem 2014; 289: 7812-7824 ([11])).

### Methodology

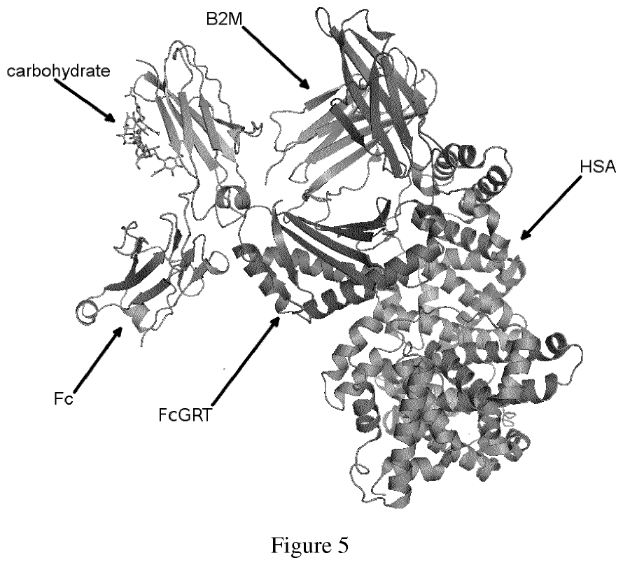

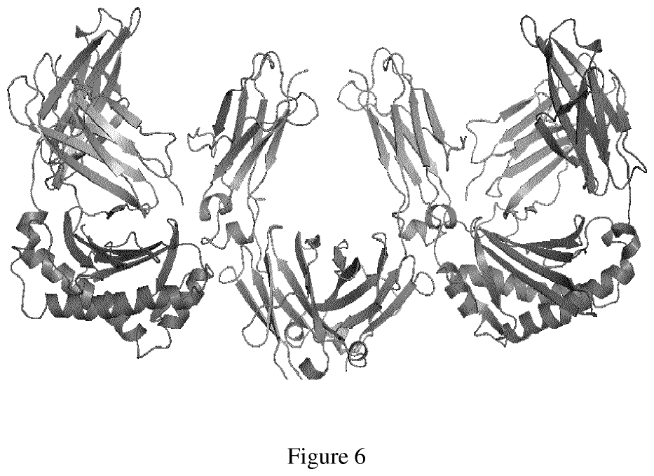

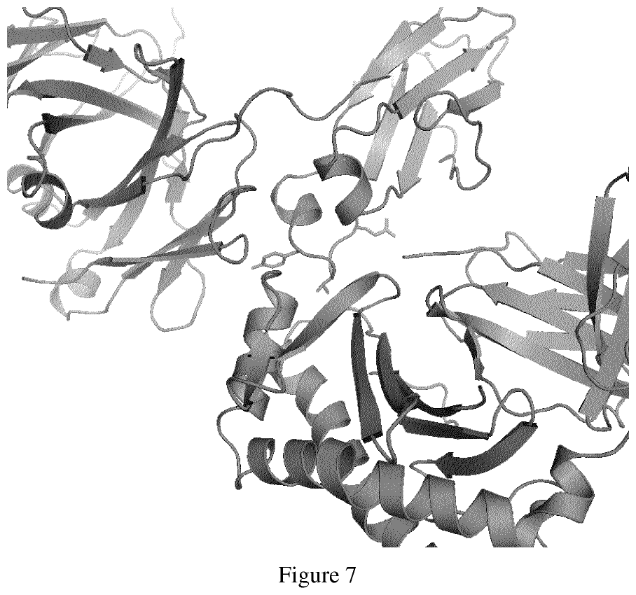

3.8 Å resolution crystal structure of the YTE human Fc variant in complex with human FcRn and human serum albumin (PDB ID 4NOU) was retrieved from the IMGT 3D database (Lefranc et al. ([4])), as opposed to the PDB, in order to ensure consistent residue numbering during any subsequent comparison with different structures. The asymmetric unit of the crystal structure contains a complex consisting of half of the Fc, bound to FcRn and HSA. However, the biological unit actually consists of the complete Fc dimer with each Fc monomer complexed with a copy of FcRn and HSA. The complete biological unit, minus the HSA component, was the subject of the modeling in order to preserve, via the dimer contacts, the conformation of the complete Fc dimer. See FIGS. 5, 6 and 7.

The canine Fc/FcRn complex contains 123 individual amino acid replacements relative to the human. In addition there are one deletion and two contiguous insertions distant from the Fc/FcRn interface; for simplicity these latter were not modeled. Notably, there are two instances in which histidine residues are mutated in the transition from the human to canine sequence, and four instances in which non-histidine residues are mutated into histidine. Of the mutated histidine residues, none are involved in significant cross-interface interactions; the same is true of the residues mutated to histidines. In the feline case the FcRn sequence incorporates a deletion relative to the human template at L(A1005), that is situated immediately behind the Fc/FcRn binding interface.

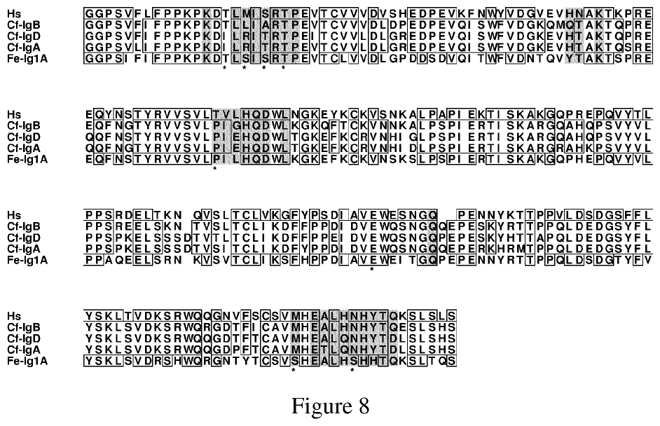

FIG. 8 presents a multiple sequence alignment encompassing the IgG sequences modeled in this study. For reference, the sections of the canine sequence that are mutually identical are boxed, the sections of the human sequence that are identical between human and canine are boxed on the human sequence, and the sections of the feline sequence that are identical between all three species are boxed on the feline sequence. The residues identified as belonging to the binding interface of the native Fc/FcRn complex (based upon the union of the ca-IgG/FcRn interface sets) are shaded. Finally the locations of the seven residues involved in the five mutation groups are marked with stars.

From the initial modeling templates, for each species-specific IgG isotype and corresponding FcRn, final models were generated: one model corresponding to the native/wild-type Fc/FcRn complex, and 1 model corresponding to the YTE mutation group. For these models, selected residues are mutated, and the rotomeric conformations of the side chains of the new amino acids and their neighboring residues are iteratively selected, based upon the avoidance of steric clashes and the possibility of favorable residue-residue interactions, from the backbone dependent rotamer library in PyMol.

### Results

The YTE mutation group consists of the substitutions L(E15A)Y, A(E16)T, and T(E18)E. Y(E15A) stabilizes the interface by desolvating P(A1050), T(E16) engages in stabilizing electrostatic and hydrogen-bonding interactions with E(A1051), and E(E18) engages in stabilizing electrostatic and hydrogen-bonding interactions with N(A1029).

Complexes of canine IgG2 and IgG4 containing the YTE mutation group are predicted to bind more tightly than those containing the native Fc; this is also consistent with the observations from the human IgG1. For ca-IgG2 the benefit of the YTE mutation can be rationalized by the following: a) L(E15A)Y gaining stabilization from P(A1050) and E(A1051), b) A(E16)T gaining stabilization from Y(A1003) and E(A1051), and c) T(E18)E gaining stabilization from N(A1029).

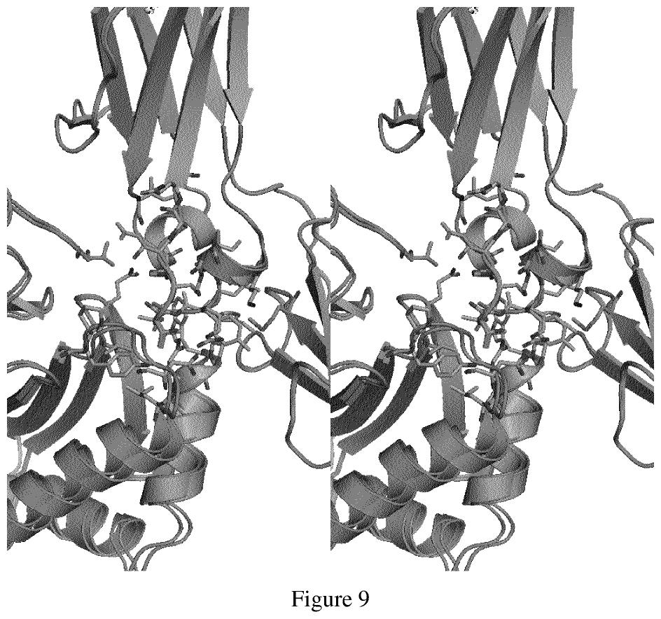

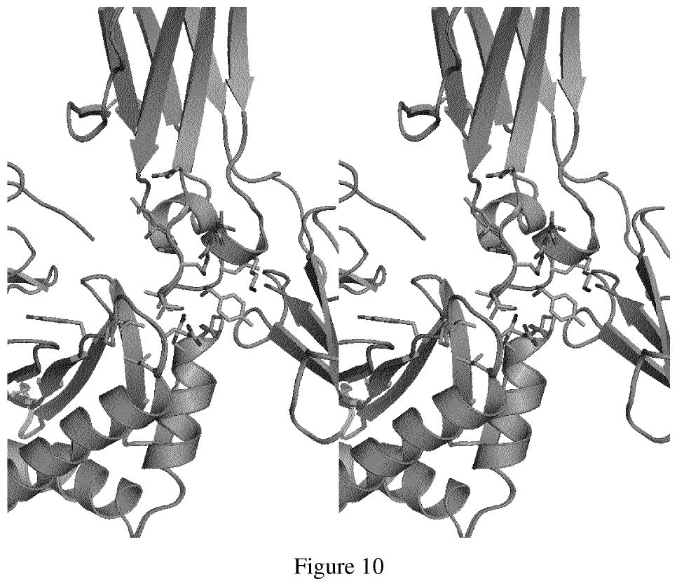

The cases of ca-IgG1 and ca-IgG4 are different because their sequence means that YTE takes the form R(E15A)Y; T(E16)T; T(E18)E. For these two cases there is no change at E16, and the complication that R(E15A) in the native derives stabilization from D(A83); this was not the situation in the ca-IgG2 with L(E15A). In the case of ca-IgG1; the R(E15A)Y results in essentially no net stabilization gain for the YTE mutation group. The environment of the ca-IgG4 YTE mutation group is illustrated in FIG. 9. FIG. 10 illustrates the environment of the feline-IgG1 YTE mutation group, S(E15A)Y; S(E16)T; T(E18)E. The figure clearly shows why the YTE mutation group does not stabilize the feline-IgG1 Fc/FcRn complex. Y(A1003) adopts a conformation directed away from the binding interface toward the FcRn chains; Thus, the models indicate that the (A1005) deletion proved to have more than a “second shell” effect; rather it directly altered the conformation of crucial contact residues. Also, the conformation of E(A1051) differs, probably due to the altered conformation of Y(A1003). Thus, there is no possibility of stabilization gains afforded by either of these residues in the presence of the YTE mutation group.

### REFERENCES

[1] Ghetie et al.: “Increasing the serum persistence of an IgG fragment by random mutagenesis”, Nat Biotechnol. 1997 July; 15(7):637-40.
[2] Dall'Acqua et al.: “Increasing the affinity of a human IgG1 for the neonatal Fc receptor: biological consequences”, J Immunol. 2002 Nov. 1; 169(9):5171-80.
[3] Tang et al.: “Cloning and characterization of cDNAs encoding four different canine immunoglobulin gamma chains”, Vet Immunol Immunopathol. 2001 Aug. 10; 80(3-4):259-70.
[4] Lefranc M. et al.: “IMGT®, the international ImMunoGeneTics information system® 25 years on”, Nucleic Acids Res 2015; 43: D413-22.
[5] Lefranc M-P: “Unique database numbering system for immunogenetic analysis”, Immunol Today 1997; 18:509.
[6] Harlow et al.: “Antibodies: A Laboratory Manual”, Cold Spring HarborLaboratory Press, 2nd ed. 1988.
[7] Hammerling, et al.: “Monoclonal Antibodies and T-Cell Hybridomas”, Elsevier, N.Y., 1981, pp. 563-681.
[8] Zoller and Smith: “Oligonucleotide-directed mutagenesis using M13-derived vectors: an efficient and general procedure for the production of point mutations in any fragment of DNA”, Nucleic Acids Res. 1982 Oct. 25; 10(20):6487-500.
[9] Kunkel et al.: “Rapid and efficient site-specific mutagenesis without phenotypic selection”, Methods Enzymol. 1987; 154:367-82.
[10] Zhou T et al.: “Structural definition of a conserved neutralization epitope on HIV-1 gp120” Nature. 2007 Feb. 15; 445(7129):732-7.
[11] Oganesyan V et al.: “Structural insights into neonatal fc receptor-based recycling mechanisms”, J Biol Chem 2014; 289: 7812-7824.
[12] The PyMOL Molecular Graphics System, Version 1.3r1, Schrodinger, LLC.

1. A canine IgG Fc domain, having an amino acid sequence comprising at least one mutation selected among:
the substitution of amino acid 15.1 of CH2 domain according to the IMGT numbering system for C-domain with tyrosine,
the substitution of amino acid 16 of CH2 domain according to the IMGT numbering system for C-domain with threonine, and
the substitution of amino acid 18 of CH2 domain according to the IMGT numbering system for C-domain with glutamic acid.

2. (canceled)

3. (canceled)

4. The canine IgG Fc domain according to claim 1, wherein said canine IgG Fc domain is selected among dog IgG2, dog IgG3 and dog IgG4.

5. The canine IgG Fc domain according to claim 1, wherein the canine IgG Fc domain is a dog (Canis lupus familiaris) IgG Fc domain.

6. The canine IgG Fc domain according to claim 1, wherein the canine IgG Fc domain includes amino acid sequence SEQ ID NO:1 or SEQ ID NO. 2.

7. The canine IgG Fc domain according to claim 1, wherein the amino acid sequence of the canine IgG Fc domain comprises:
the substitution of amino acid 15.1 of CH2 domain according to the IMGT numbering system for C-domain with tyrosine,
the substitution of amino acid 16 of CH2 domain according to the IMGT numbering system for C-domain with threonine, and
the substitution of amino acid 18 of CH2 domain according to the IMGT numbering system for C-domain with glutamic acid.

8. An Fc-fusion protein, comprising a canine IgG Fc domain according to claim 1, that is genetically linked to a peptide a protein, an engineered ligand-binding proteins or a VHH domain.

9. An antibody comprising a canine IgG Fc domain according to claim 1.

10. (canceled)

11. (canceled)

12. (canceled)

13. The antibody according to claims 9, wherein the antibody binds to at least one of:
(a) an epitope selected among IL-31, IL31R, IL13, IL4, IL4R, IL13R, IL-5, IL23, IL22, IGF-1, CCL17, CD14, CD-20, CD-52 , CD40, CD50, CD80, CD154, CD163, CX3CL1, CCR2, CXCR2, CGRP, CHST14 antibodies, TNF-α, TNFR1 HER-1, HER-2, Ig-E, NGF, PD-1, PD-L1, Nav1.3, Nav1.5, Nav1.7, TSLP, TGF-β, p53 protein, Flt3 ligand, GM-CSF, protein or peptide of myelin oligodendrocytes glycoprotein, MMP-13, MMP-3, MMP-1, ADAMTS-4, ADAMTS-5, uPA, uPAR, soluble receptor involved in blockade activation or modulation of innate immunity or adaptive immunity related to inflammatory disease, for example TSLPR or TARC, 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAMS, ADAMTS, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin 0, Cathepsin S, Cathepsin V, Cathepsin X/ZIP, CBL, CCI, CCK2, CCL, CCL1, CCL11, CCL12, CCL13, CCL14, CCL15, CCL16, CCL18, CCL19, CCL2, CCL20, CCL21, CCL22, CCL23, CCL24, CCL25, CCL26, CCL27, CCL28, CCL3, CCL4, CCLS, CCL6, CCL7, CCL8, CCL9/10, CCR, CCR1, CCR10, CCR10, CCR3, CCR4, CCRS, CCR6, CCR7, CCR8, CCR9, CD1, CD2, CD3, CD3E, CD4, CDS, CD6, CD7, CD8, CD10, CD11a, CD11b, CD11c, CD13, CD15, CD16, CD18, CD19, CD21, CD22, CD23, CD25, CD27L, CD28, CD29, CD30, CD30L, CD32, CD33 (p67 proteins), CD34, CD38, CD40L, CD44, CD45, CD46, CD49a, CD54, CD55, CD56, CD61, CD64, CD66e, CD74, CD89, CD95, CD123, CD137, CD138, CD140a, CD146, CD147, CD148, CD152, CD164, CEACAMS, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CX3CR1, CXCL, CXCL1, CXCL2, CXCL3, CXCL4, CXCLS, CXCL6, CXCL7, CXCL8, CXCL9, CXCL10, CXCL11, CXCL12, CXCL13, CXCL14, CXCL15, CXCL16, CXCR, CXCR1, CXCR3, CXCR4, CXCRS, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, Enkephalinase, eNOS, Eot, eotaxinl, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MM), HIV gp120, HIV IIIB gp120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IGF, IGF binding proteins, IGF-1R, IGFBP, IGF-I, IGF-II, IL, IL-1, IL-1R, IL-2, IL-2R, IL-5, IL-5R, IL-6, IL-6R, IL-8, IL-9, IL-10, IL-12, IL-15, IL-18, IL-18R, interferon (INF)-alpha, INF-beta, INF-gamma, Inhibin, iNOS, Insulin A-chain, Insulin B-chain, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bp1, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MP-1-alpha, MK, MMAC1, MMP, MMP-1, MMP-10, MMP-11, MMP-12, MMP-14, MMP-15, MMP-2, MMP-24, MMP-3, MMP-7, MMP-8, MMP-9, MPIF, Mpo, MSK, MSP, mucin (Mucl), MUC18, Muellerian-inhibitin substance, Mug, MuSK, NAIP, NAP, NCAD, N-Cadherin, NCA 90, NCAM, NCAM, Neprilysin, Neurotrophin-3, -4, or -6, Neurturin, NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGD2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, 5100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha beta, TNF-beta2, TNFc, TNF-RII, TNFRSF10A (TRAIL R1 Apo-2, DR4), TNFRSF10B (TRAIL R2 DRS, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF RI CD120a, p55-60), TNFRSF1B (TNF RII CD120b, p′75-80), TNFRSF26 (TNFRH3), TNFRSF3 (LTbR TNF RIII, TNFC R), TNFRSF4 (OX40 ACT35, TXGP1 R), TNFRSFS (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSFS (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, tumor-associated antigen CA 125, tumor-associated antigen expressing Lewis Y related carbohydrate, TWEAK, TXB2, Ung, uPAR-1, Urokinase, VCAM, VCAM-1, VECAD, VE-Cadherin, VE-cadherin-2, VEFGR-1 (flt-1), VEGF, VEGFR, VEGFR-3 (fit-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and receptors for hormones and growth factors;
(b) a parasite epitope is selected in the group comprising an exposed antigen including T-cell activation inhibitors for example p36, Iris, Salp15 and IL-2 binding protein, 64P tick protein, Salp15, Salp25D, HL34, P29, RIM36, Caltericulin, Tick Histamine Release Factor (tHRF) and AamAV422, a concealed antigen for example Bm86 protein, ferritins for example ferritin 2, HIFER1 and HIFER2, serpins (Serine protease inhibitors) for example RAS-3, RAS-4 and RIM36, 4D8, Subolesin (SUB)/Akirin, HLS1 serine proteinase inhibitor, HLS2 serine proteinase inhibitor, P27/P30 troponin I-like protein, caltericulin, Bm91, voraxin, peritrophin, akirins, midgut mucins, Manα1-6 proximal to Galβ1-4GlcNAc-α-O-R glycans, Maxadilan factor (MAX), Salivary Gland Lysate (SGL), Salivary Gland Protein 15 (SP15), microfilarial IgM-activating antigens for example polypeptide P34, polypeptide P38, 14-16 kDa microfilarial antigens, 63 kDa microfilarial antigen or 73 kDA microfilarial antigen, microfilarial IgG-activating antigens for example 36kDa antigen, 38 kDa antigen, 71 kDa antigen or 84 kDa antigen, third stage larval antigens for example polypeptides P200, P130, P100, P80, P75, P38, P34, P32, P21, P15, 14 kDa antigen, 20 kDa antigen, 30 kDa antigen, 34 kDa antigen, 35 kDa major surface antigen or 39 kDa antigen, fourth stage larval antigens for example 39 kDa antigen, 66 kDa antigen, 24/23 kDa doublet antigen, 15 kDa antigen, 31 kDa antigen, 39 kDa antigen, 42 kDa antigen, 55 kDa antigen, 59 kDa antigen, 70 kDa antigen, 97 kDa antigen or 207 kDa antigen, adult stage antigens for example 15 kDa antigen, 20 kDa antigen or 38 kDa antigen, and a universal antigen for example DiAg, Di5 (cuticular) antigen, somatic antigens for example tropomyosin, major sperm protein, P22U or small heat shock protein 12.6, a surface antigen for example papain-like cysteine proteinase or GADPH (Glyceraldehyde 3-phosphate dehydrogenase), Excretory-Secretory (E/S) products for example Triose Phosphate Isomerase, Heat Shock Protein 70 (HSP70) and Transthyretin;
(c) a pathogens or pathogen-derived material for example lipopolysaccharides, peptidoglycans, cell wall components, cell membrane components, toxins, siderophores, virulence factors, adhesins or molecules involved in quorum sensing, ceceptors involved in activation, blockade or modulation of innate immunity or adaptive immunity, for example Pattern Recognition Receptors like Toll-like receptors or C-type Lectin Receptors, G-Protein Coupled Receptors, costimulatory membrane proteins or immune checkpoint inhibitors such as B7 protein family, Programmed cell Death molecules (PD) and PD ligands (PD-L), CTLA-4, or LAG-3, and cytokines for example chemokines, interleukins, interferons, mediators involved in promotion or resolution of inflammation, in activation, blockade or modulation of innate immunity or adaptive immunity; or
a combination thereof.

14. The Fc-fusion protein according to claims 8, wherein the Fc-fusion protein binds to at least one of:
(a) an epitope selected among IL-31, IL31R, IL13, IL4, IL4R, IL13R, IL-5, IL23, IL22, IGF-1, CCL17, CD14, CD-20, CD-52 , CD40, CD50, CD80, CD154, CD163, CX3CL1, CCR2, CXCR2, CGRP, CHST14 antibodies, TNF-α, TNFR1 HER-1, HER-2, Ig-E, NGF, PD-1, PD-L1, Nav1.3, Nav1.5, Nav1.7, TSLP, TGF-β, p53 protein, Flt3 ligand, GM-CSF, protein or peptide of myelin oligodendrocytes glycoprotein, MMP-13, MMP-3, MMP-1, ADAMTS-4, ADAMTS-5, uPA, uPAR, soluble receptor involved in blockade activation or modulation of innate immunity or adaptive immunity related to inflammatory disease, for example TSLPR or TARC, 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAMS, ADAMTS, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Ax1, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bc1, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin 0, Cathepsin S, Cathepsin V, Cathepsin X/ZIP, CBL, CCI, CCK2, CCL, CCL1, CCL11, CCL12, CCL13, CCL14, CCL15, CCL16, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD15, CD16, CD18, CD19, CD21, CD22, CD23, CD25, CD27L, CD28, CD29, CD30, CD30L, CD32, CD33 (p67 proteins), CD34, CD38, CD40L, CD44, CD45, CD46, CD49a, CD54, CD55, CD56, CD61, CD64, CD66e, CD74, CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, Enkephalinase, eNOS, Eot, eotaxinl, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MM), HIV gp120, HIV IIIB gp120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IGF, IGF binding proteins, IGF-1R, IGFBP, IGF-I, IGF-II, IL, IL-1, IL-1R, IL-2, IL-2R, IL-5, IL-5R, IL-6, IL-6R, IL-8, IL-9, IL-10, IL-12, IL-15, IL-18, IL-18R, interferon (INF)-alpha, INF-beta, INF-gamma, Inhibin, iNOS, Insulin A-chain, Insulin B-chain, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bp1, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MP-1-alpha, MK, MMAC1, MMP, MMP-1, MMP-10, MMP-11, MMP-12, MMP-14, MMP-15, MMP-2, MMP-24, MMP-3, MMP-7, MMP-8, MMP-9, MPIF, Mpo, MSK, MSP, mucin (Mucl), MUC18, Muellerian-inhibitin substance, Mug, MuSK, NAIP, NAP, NCAD, N-Cadherin, NCA 90, NCAM, NCAM, Neprilysin, Neurotrophin-3, -4, or -6, Neurturin, NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGD2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, 5100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha beta, TNF-beta2, TNFc, TNF-RII, TNFRSF10A (TRAIL R1 Apo-2, DR4), TNFRSF10B (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF RI CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, tumor-associated antigen CA 125, tumor-associated antigen expressing Lewis Y related carbohydrate, TWEAK, TXB2, Ung, uPAR-1, Urokinase, VCAM, VCAM-1, VECAD, VE-Cadherin, VE-cadherin-2, VEFGR-1 (flt-1), VEGF, VEGFR, VEGFR-3 (fit-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and receptors for hormones and growth factors;
(b) binding to a parasite epitope is selected in the group comprising an exposed antigen including T-cell activation inhibitors for example p36, Iris, Salp15 and IL-2 binding protein, 64P tick protein, Salp15, Salp25D, HL34, P29, RIM36, Caltericulin, Tick Histamine Release Factor (tHRF) and AamAV422, a concealed antigen for example Bm86 protein, ferritins for example ferritin 2, HIFER1 and HIFER2, serpins (Serine protease inhibitors) for example RAS-3, RAS-4 and RIM36, 4D8, Subolesin (SUB)/Akirin, HLS1 serine proteinase inhibitor, HLS2 serine proteinase inhibitor, P27/P30 troponin I-like protein, caltericulin, Bm91, voraxin, peritrophin, akirins, midgut mucins, Manα1-6 proximal to Galβ1-4GlcNAc-α-O-R glycans, Maxadilan factor (MAX), Salivary Gland Lysate (SGL), Salivary Gland Protein 15 (SP15), microfilarial IgM-activating antigens for example polypeptide P34, polypeptide P38, 14-16 kDa microfilarial antigens, 63 kDa microfilarial antigen or 73 kDA microfilarial antigen, microfilarial IgG-activating antigens for example 36kDa antigen, 38 kDa antigen, 71 kDa antigen or 84 kDa antigen, third stage larval antigens for example polypeptides P200, P130, P100, P80, P75, P38, P34, P32, P21, P15, 14 kDa antigen, 20 kDa antigen, 30 kDa antigen, 34 kDa antigen, 35 kDa major surface antigen or 39 kDa antigen, fourth stage larval antigens for example 39 kDa antigen, 66 kDa antigen, 24/23 kDa doublet antigen, 15 kDa antigen, 31 kDa antigen, 39 kDa antigen, 42 kDa antigen, 55 kDa antigen, 59 kDa antigen, 70 kDa antigen, 97 kDa antigen or 207 kDa antigen, adult stage antigens for example 15 kDa antigen, 20 kDa antigen or 38 kDa antigen, and a universal antigen for example DiAg, Di5 (cuticular) antigen, somatic antigens for example tropomyosin, major sperm protein, P22U or small heat shock protein 12.6, a surface antigen for example papain-like cysteine proteinase or GADPH (Glyceraldehyde 3-phosphate dehydrogenase), Excretory-Secretory (E/S) products for example Triose Phosphate Isomerase, Heat Shock Protein 70 (HSP70) and Transthyretin;
(c) a pathogens or pathogen-derived material for example lipopolysaccharides, peptidoglycans, cell wall components, cell membrane components, toxins, siderophores, virulence factors, adhesins or molecules involved in quorum sensing, ceceptors involved in activation, blockade or modulation of innate immunity or adaptive immunity, for example Pattern Recognition Receptors like Toll-like receptors or C-type Lectin Receptors, G-Protein Coupled Receptors, costimulatory membrane proteins or immune checkpoint inhibitors such as B7 protein family, Programmed cell Death molecules (PD) and PD ligands (PD-L), CTLA-4, or LAG-3, and cytokines for example chemokines, interleukins, interferons, mediators involved in promotion or resolution of inflammation, in activation, blockade or modulation of innate immunity or adaptive immunity; or
a combination thereof.

15. (canceled)

16. A method of treating or preventing a disease or disorder, the method comprising administering a therapeutic to a subject in need thereof,
wherein:
the therapeutic comprises: (1) the canine IgG Fc domain according to claim 1, (2) an Fc-fusion protein comprising the canine IgG Fc domain of (1) that is genetically linked to a peptide, a protein, an engineered ligand-binding protein, or a VHH domain, or (3) an antibody comprising the canine IgG Fc domain of (1); and
the therapeutic is effective at treating or preventing the disease or disorder.

17. (canceled)

18. The method according to claim 16, wherein the therapeutic is a vaccine.

19. The method according to claim 16, wherein the therapeutic facilitates delivery of a protein, across an epithelial barrier or a mucosal barrier.

20. The method according to claim 16, wherein the disease or disorder is selected from an inflammatory disease, an auto-immune disease or disorder, an IgG mediated autoimmune disease, an immune-mediated disease, osteoarthritis, atopic dermatitis, skin inflammatory disease, otitis, infectious disease, and respiratory disease.

21. The method according to claim 204, wherein:
said autoimmune disease is selected among from bullous autoimmune skin disease, systemic lupus erythematosus, autoimmune hemolytic anemia, immune-mediated thrombocytopenia, thrombocytopenia, autoimmune blood disease, autoimmune musculoskeletal system disease, autoimmune thyroid disease, multiple organ autoimmune diseases, autoimmune adrenal gland autoimmune disease and hypothyroidism.

22. The method according to claim 21, wherein:
said bullous autoimmune skin disease includes pemphigus vulgaris, pemphigus foliaceus, pemphigus vegetans, pemphigus erythematosus and bullous pemphigoid thyroid;
said autoimmune blood disease is selected among autoimmune haemolytic anaemia, immune-mediated thrombocytopenia and systemic lupus erythematosus; or
said autoimmune musculoskeletal system disease is selected among myasthenia gravis, rheumatoid arthritis, systemic lupus erythematosus and polyarthritis;
said autoimmune thyroid disease is associated with lymphocytic thyroiditis;
said multiple organ autoimmune diseases is selected among systemic lupus erythematosus and discoid lupus erythematosus; or
said autoimmune adrenal gland autoimmune disease is hypoadrenocorticism.

23. (canceled)

24. (canceled)

25. (canceled)

26. (canceled)

27. The method according to claim 28, wherein the inflammatory diseases is selected from atopic dermatitis, osteoarthrosis, or an allergy.

28. The method according to claim 20, wherein said inflammatory disease is a skin inflammatory disease, an osteo-joint disease, cancer, or immune disease.

29. The method according to claim 16, wherein the disease or the disorder is an infectious disease or a parasitic disease.

30. The method according to claim 29, wherein said infectious or parasitic diseases are selected from:
a disease induced by an ectoparasite of a dog or an endoparasite of a dogs;
a respiratory infection;
a urinary infection; and
a dermatological infection.

31. The method according to claim 30, wherein:
the ectoparasite or endoparasite is selected from: ticks (Arachnida: Ixodida), mites (Arachnida: Acari), chewing and biting lice (Arthropoda: Phthiraptera), fleas (Arthropoda: Siphonaptera), flies (Diptera: Nematocera and Brachycera), mosquitoes (Diptera: Culicidae), sand flies (Diptera: Psychodidae), nematodes (Nemathelminthes: Nematoda), trematodes (Plathelminthes: Trematoda), cestodes (Plathelminthes: Cestoda) and protozoa (Protista: Protozoa)
the dermatological infention is selected from skin infection, soft tissue invection, and otitis;
said infectious respiratory infections are selected among diseases induced by Bordetella bronchiseptica, Mycoplasma spp (M. canis, M. cynos), Streptococcus spp, Escherichia coli, Pasteurella multocida, Staphylococcus spp, CIV/Canine influenza virus, CPIV/canine parainfluenza virus, CnPnV/Canine pneumovirus, CDV/Canin distemper virus, CRCoV/Canine respiratory coronavirus, CAdV-2/Canine adenovirus type 2, and CaHV-1/Canine herpesvirus type 1;
the urinary infection is selected from diseases induced by Staphylococcus pseudintermedius, Staphylococcus aureus, Coagulase-negative staphylococcus spp, Pseudomonas aeruginosa, Proteus spp, Escherichia coli, Corynebacterium spp, Enterococcus spp, Citrobacter spp, Enterobacter spp, Mycoplasma spp, Lactobacillus spp, Klebsiella spp, Anaerobic bacteria;
the skin and soft tissues infection is selected from diseases induced by Staphylococcus pseudintermedius, Staphylococcus aureus, Pseudomonas aeruginosa, Proteus spp, Escherichia coli, Corynebacterium spp, Enterococcus spp, Citrobacter spp, Lactobacillus spp, Klebsiella spp, Anaerobic bacteria, Malassezia pachydermatis, and Malassezia spp.

32. (canceled)

33. (canceled)

34. A diagnostic method comprising contacting a sample with (1) the canine IgG Fc domain according to claim 1, (2) an Fc-fusion protein comprising the canine IgG Fc domain of (1) that is genetically linked to a peptide, protein, engineered ligand-binding protein, or a VHH domain, or (3) an antibody comprising the canine IgG Fc domain of (1).

35. (canceled)

36. A nucleic acid comprising a sequence that encodes for (1) the canine IgG Fc domain according to claim 1, (2) an Fc-fusion protein comprising the canine IgG Fc domain of (1) that is genetically linked to a peptide, a protein, an engineered ligand-binding protein, or a VHH domain, or (3) an antibody comprising the canine IgG Fc domain of (1).

37. An expression vector having the nucleic acid as defined inof claim 36.

38. A stable cell line producing a canine IgG Fc domain, an Fc-fusion protein, or an antibody, the stable cell line having the expression vector of claim 37.

39. (canceled)

40. An in vitro process for producing (1) a canine IgG Fc domain, (2) an Fc-fusion protein, or (3) an antibody, the method comprising the steps of:
(A) providing a host cell with an expression vector having the nucleic acid of claim 36, and culturing the host cell under conditions such that the host cell expresses said nucleic acid, and,
(B) collecting the canine IgG Fc domain, the Fc-fusion protein, or the antibody produced by the host cell.

41. A method for increasing the binding affinity of a canine IgG Fc region for the canine FcRn, compared to the corresponding wild type canine IgG Fc domain, said method comprising modifying a canine IgG Fc domain to include at least one mutation selected from:
the substitution of amino acid 15.1 of CH2 domain according to the IMGT numbering system for C-domain with tyrosine,
the substitution of amino acid 16 of CH2 domain according to the IMGT numbering system for C-domain with threonine, and
the substitution of amino acid 18 of CH2 domain according to the IMGT numbering system for C-domain with glutamic acid.

42. A method for increasing the in vivo half-life of a canine antibody or an Fc fusion protein, compared to the corresponding wild type canine antibody or Fc fusion protein, said method comprising modifying the canine IgG Fc domain of said antibody to include at least one mutation selected from:
the substitution of amino acid 15.1 of CH2 domain according to the IMGT numbering system for C-domain with tyrosine,
the substitution of amino acid 16 of CH2 domain according to the IMGT numbering system for C-domain with threonine, and
the substitution of amino acid 18 of CH2 domain according to the IMGT numbering system for C-domain with glutamic acid.

43. A composition comprising:
a pharmaceutically acceptable carrier, a pharmaceutically acceptable excipient, a pharmaceutically acceptable stabilizer, or a combination thereof; and
(1) the canine IgG Fc domain according to claim 1, (2) an Fc-fusion protein comprising the canine IgG Fc domain of (1) that is genetically linked to a peptide, a protein, an engineered ligand-binding protein, or a VHH domain, or (3) an antibody comprising the canine IgG Fc domain of (1).


SEQUENCE LISTING (6 sequences)

SEQ ID NO: 1 - protein, 219 aa, Canis familiaris
APEMLGGPSVFIFPPKPKDTLYITREPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 2 - protein, 220 aa, Canis familiaris
VPESLGGPSVFIFPPKPKDILYITREPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 3 - protein, 236 aa, Canis familiaris
KRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 4 - protein, 233 aa, Canis familiaris
PKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 5 - protein, 18 aa, Artificial Sequence
  Synthetic
MGVPTQLGLLLWLTDARC

SEQ ID NO: 6 - protein, 19 aa, Artificial Sequence
  Synthetic
MEWSWVFLFFLSVTTGVHS